SEQUENCE LISTING (116 sequences)

SEQ ID NO: 1 - protein, 7 aa, Homo sapiens
VLAAVYK

SEQ ID NO: 2 - protein, 12 aa, Homo sapiens
SLAPYAQDTQEK

SEQ ID NO: 3 - protein, 7 aa, Homo sapiens
QSELSAK

SEQ ID NO: 4 - protein, 9 aa, Homo sapiens
TAAQNLYEK

SEQ ID NO: 5 - protein, 11 aa, Homo sapiens
TSNFNAAISLK

SEQ ID NO: 6 - protein, 11 aa, Homo sapiens
LSPIYNLVPVK

SEQ ID NO: 7 - protein, 8 aa, Homo sapiens
EAFSLFDK

SEQ ID NO: 8 - protein, 10 aa, Homo sapiens
ALSIGFETCR

SEQ ID NO: 9 - protein, 8 aa, Homo sapiens
IASNTQSR

SEQ ID NO: 10 - protein, 8 aa, Homo sapiens
GEFYIGSK

SEQ ID NO: 11 - protein, 7 aa, Homo sapiens
VDAASLK

SEQ ID NO: 12 - protein, 8 aa, Homo sapiens
EFFVGLSK

SEQ ID NO: 13 - protein, 10 aa, Homo sapiens
DQDGEILLPR

SEQ ID NO: 14 - protein, 8 aa, Homo sapiens
IEEELGSK

SEQ ID NO: 15 - protein, 8 aa, Homo sapiens
LNVTEQEK

SEQ ID NO: 16 - protein, 10 aa, Homo sapiens
IEEELGDEAR

SEQ ID NO: 17 - protein, 8 aa, Homo sapiens
YTACETAR

SEQ ID NO: 18 - protein, 7 aa, Homo sapiens
AAFNSGK

SEQ ID NO: 19 - protein, 6 aa, Homo sapiens
YDNSLK

SEQ ID NO: 20 - protein, 11 aa, Homo sapiens
DHLLGVSDSGK

SEQ ID NO: 21 - protein, 8 aa, Homo sapiens
IGADFLAR

SEQ ID NO: 22 - protein, 13 aa, Homo sapiens
AGALNSNDAFVLK

SEQ ID NO: 23 - protein, 7 aa, Homo sapiens
VQVVAGK

SEQ ID NO: 24 - protein, 8 aa, Homo sapiens
GQLSFNLR

SEQ ID NO: 25 - protein, 7 aa, Homo sapiens
VWVQAFK

SEQ ID NO: 26 - protein, 9 aa, Homo sapiens
YLDFVFAVK

SEQ ID NO: 27 - protein, 8 aa, Homo sapiens
FIIPQIVK

SEQ ID NO: 28 - protein, 10 aa, Homo sapiens
GEFVTTVQQR

SEQ ID NO: 29 - protein, 12 aa, Homo sapiens
GLGEISAASEFK

SEQ ID NO: 30 - protein, 8 aa, Homo sapiens
VAELEDEK

SEQ ID NO: 31 - protein, 8 aa, Homo sapiens
ELDVLQGR

SEQ ID NO: 32 - protein, 10 aa, Homo sapiens
LAIGFSTVQK

SEQ ID NO: 33 - protein, 10 aa, Homo sapiens
LEGNPIVLGK

SEQ ID NO: 34 - protein, 10 aa, Homo sapiens
LESLPAHLPR

SEQ ID NO: 35 - protein, 7 aa, Homo sapiens
ALVILAK

SEQ ID NO: 36 - protein, 8 aa, Homo sapiens
VLTPTQVK

SEQ ID NO: 37 - protein, 8 aa, Homo sapiens
TFTLLDPK

SEQ ID NO: 38 - protein, 7 aa, Homo sapiens
VVEVGSK

SEQ ID NO: 39 - protein, 7 aa, Homo sapiens
LFEELVR

SEQ ID NO: 40 - protein, 9 aa, Homo sapiens
LLIGTVFHK

SEQ ID NO: 41 - protein, 9 aa, Homo sapiens
VSFELFADK

SEQ ID NO: 42 - protein, 11 aa, Homo sapiens
AIIDGVESVSR

SEQ ID NO: 43 - protein, 10 aa, Homo sapiens
IESQTQEEVR

SEQ ID NO: 44 - protein, 8 aa, Homo sapiens
AQALEQAK

SEQ ID NO: 45 - protein, 12 aa, Homo sapiens
HVGDLGNVTADK

SEQ ID NO: 46 - protein, 7 aa, Homo sapiens
QETLPSK

SEQ ID NO: 47 - protein, 10 aa, Homo sapiens
IAVAAQNCYK

SEQ ID NO: 48 - protein, 12 aa, Homo sapiens
EPVAGDAVPGPK

SEQ ID NO: 49 - protein, 9 aa, Homo sapiens
GLQEAAEER

SEQ ID NO: 50 - protein, 10 aa, Homo sapiens
GQYCYELDEK

SEQ ID NO: 51 - protein, 8 aa, Homo sapiens
NLLSVAYK

SEQ ID NO: 52 - protein, 8 aa, Homo sapiens
VISSIEQK

SEQ ID NO: 53 - protein, 8 aa, Homo sapiens
VVSSIEQK

SEQ ID NO: 54 - protein, 10 aa, Homo sapiens
CLAVYQAGAR

SEQ ID NO: 55 - protein, 9 aa, Homo sapiens
LGADMEDVR

SEQ ID NO: 56 - protein, 8 aa, Homo sapiens
ELQAAQAR

SEQ ID NO: 57 - protein, 11 aa, Homo sapiens
LGADMEDVCGR

SEQ ID NO: 58 - protein, 9 aa, Homo sapiens
LAVYQAGAR

SEQ ID NO: 59 - protein, 10 aa, Homo sapiens
LVNEVTEFAK

SEQ ID NO: 60 - protein, 7 aa, Homo sapiens
LVTDLTK

SEQ ID NO: 61 - protein, 12 aa, Homo sapiens
FLASVSTVLTSK

SEQ ID NO: 62 - protein, 13 aa, Homo sapiens
VNVDEVGGEALGR

SEQ ID NO: 63 - protein, 10 aa, Homo sapiens
VTSGSTSTSR

SEQ ID NO: 64 - protein, 8 aa, Homo sapiens
LASVSVSR

SEQ ID NO: 65 - protein, 10 aa, Homo sapiens
YVYVADVAAK

SEQ ID NO: 66 - protein, 9 aa, Homo sapiens
VVGGLVALR

SEQ ID NO: 67 - protein, 10 aa, Homo sapiens
LLSLGAGEFK

SEQ ID NO: 68 - protein, 9 aa, Homo sapiens
LGFTDLFSK

SEQ ID NO: 69 - protein, 8 aa, Homo sapiens
AAQEEYVK

SEQ ID NO: 70 - protein, 7 aa, Homo sapiens
ADQDTIR

SEQ ID NO: 71 - protein, 7 aa, Homo sapiens
DGADFAK

SEQ ID NO: 72 - protein, 12 aa, Homo sapiens
DGNGYISAAELR

SEQ ID NO: 73 - protein, 12 aa, Homo sapiens
DIEEGAIVNPGR

SEQ ID NO: 74 - protein, 9 aa, Homo sapiens
DYSVTANSK

SEQ ID NO: 75 - protein, 10 aa, Homo sapiens
EGDCPVQSGK

SEQ ID NO: 76 - protein, 14 aa, Homo sapiens
EHAVEGDCDFQLLK

SEQ ID NO: 77 - protein, 8 aa, Homo sapiens
ELSDIAHR

SEQ ID NO: 78 - protein, 8 aa, Homo sapiens
ENFSCLTR

SEQ ID NO: 79 - protein, 14 aa, Homo sapiens
EPCGGLEDAVNEAK

SEQ ID NO: 80 - protein, 11 aa, Homo sapiens
ESLSSYWESAK

SEQ ID NO: 81 - protein, 11 aa, Homo sapiens
EVTGIITQGAR

SEQ ID NO: 82 - protein, 7 aa, Homo sapiens
FIVYSYK

SEQ ID NO: 83 - protein, 12 aa, Homo sapiens
FVEGLPINDFSR

SEQ ID NO: 84 - protein, 15 aa, Homo sapiens
GALQNIIPASTGAAK

SEQ ID NO: 85 - protein, 11 aa, Homo sapiens
GDLGIEIPAEK

SEQ ID NO: 86 - protein, 9 aa, Homo sapiens
GDSVVYGLR

SEQ ID NO: 87 - protein, 9 aa, Homo sapiens
GDYPLEAVR

SEQ ID NO: 88 - protein, 17 aa, Homo sapiens
GECVPGEQEPEPILIPR

SEQ ID NO: 89 - protein, 20 aa, Homo sapiens
GNDISSGTVLSDYVGSGPPK

SEQ ID NO: 90 - protein, 14 aa, Homo sapiens
GNQWVGYDDQESVK

SEQ ID NO: 91 - protein, 12 aa, Homo sapiens
GVCEETSGAYEK

SEQ ID NO: 92 - protein, 17 aa, Homo sapiens
GVNLPGAAVDLPAVSEK

SEQ ID NO: 93 - protein, 15 aa, Homo sapiens
HVLFGTVGVPEHTYR

SEQ ID NO: 94 - protein, 10 aa, Homo sapiens
HYGGLTGLNK

SEQ ID NO: 95 - protein, 11 aa, Homo sapiens
ICEPGYSPTYK

SEQ ID NO: 96 - protein, 12 aa, Homo sapiens
IVFLEEASQQEK

SEQ ID NO: 97 - protein, 11 aa, Homo sapiens
LIVHNGYCDGR

SEQ ID NO: 98 - protein, 17 aa, Homo sapiens
LLVFATDDGFHFAGDGK

SEQ ID NO: 99 - protein, 8 aa, Homo sapiens
LYEQLSGK

SEQ ID NO: 100 - protein, 12 aa, Homo sapiens
SGQLGIQEEDLR

SEQ ID NO: 101 - protein, 13 aa, Homo sapiens
TATSEYQTFFNPR

SEQ ID NO: 102 - protein, 8 aa, Homo sapiens
TEAADLCK

SEQ ID NO: 103 - protein, 14 aa, Homo sapiens
TLLSVGGWNFGSQR

SEQ ID NO: 104 - protein, 8 aa, Homo sapiens
VFEDESGK

SEQ ID NO: 105 - protein, 9 aa, Homo sapiens
VGNLTVVGK

SEQ ID NO: 106 - protein, 12 aa, Homo sapiens
VIGSGCNLDSAR

SEQ ID NO: 107 - protein, 17 aa, Homo sapiens
VIVVGNPANTNCLTASK

SEQ ID NO: 108 - protein, 15 aa, Homo sapiens
VNQIGSVTEAIQACK

SEQ ID NO: 109 - protein, 12 aa, Homo sapiens
VTLSAAPPSYFR

SEQ ID NO: 110 - protein, 9 aa, Homo sapiens
VVEGSFVYK

SEQ ID NO: 111 - protein, 15 aa, Homo sapiens
WGLGGTCVNVGCIPK

SEQ ID NO: 112 - protein, 12 aa, Homo sapiens
YGFIEGHVVIPR

SEQ ID NO: 113 - protein, 12 aa, Homo sapiens
YISPDQLADLYK

SEQ ID NO: 114 - protein, 10 aa, Homo sapiens
YLAEVATGEK

SEQ ID NO: 115 - protein, 12 aa, Homo sapiens
YLIPNATQPESK

SEQ ID NO: 116 - protein, 13 aa, Homo sapiens
YVWLVYEQDRPLK

(19) United States
(12) Patent Application Publication
(10) Pub. No.: US 2026/0126454 A1
(43) Pub. Date: May 7, 2026

(54) MASS SPECTROMETRY ANALYSIS OF MARKERS FOR ALZHEIMER'S DISEASE

(71) Applicant: Emory University, Atlanta, GA (US)

(72) Inventors: Nicholas Seyfried, Atlanta, GA (US); Duc Duong, Atlanta, GA (US); Allan Levey, Atlanta, GA (US); James Lah, Atlanta, GA (US); Caroline Watson, Atlanta, GA (US)

(73) Assignee: Emory University, Atlanta, GA (US)

(21) Appl. No.: 19/106,781

(22) PCT Filed: Aug. 25, 2023
(86) PCT No.: PCT/US2023/072936

§ 371 Date: Feb. 26, 2025

Related U.S. Application Data
(60) Provisional application No. 63/401,551, filed on Aug. 26, 2022.

(51) Int. Cl. G01N 33/68

(52) U.S. Cl. CPC G01N 33/6896; G01N 33/6842; G01N 33/6848; G01N 2570/00; G01N 2800/2821

(57) ABSTRACT

Provided herein is a sensitive, quantitative, and scalable targeted proteomics assay of Alzheimer's Disease biomarkers representing neuronal, glial, vasculature and metabolic pathways. The biomarkers are protease-digested peptides selected from biological samples of individuals having normal Aβ and Tau levels (AT−) and from symptomatic and asymptomatic individuals having low Aβ and high Tau levels (AT+). The assay uses selective reaction monitoring-based mass spectrometry (SRM-MS) of peptides in the biological samples after digestion.

### CROSS REFERENCE TO RELATED APPLICATIONS

This is the U.S. National Stage of International Application No. PCT/US2023/072936, filed Aug. 25, 2023, which was published in English under PCT Article 21 (2), which in turn claims the benefit of U.S. Provisional Application No. 63/401,551, filed on Aug. 26, 2022, the entire disclosure of each application is hereby incorporated herein by reference in its entirety for all purposes.

### STATEMENT REGARDING FEDERALLY SPONSORED RESEARCH

This invention was made with government support under Grant Nos. AG046161 and AG025688 awarded by the National Institutes of Health (NIH). The government has certain rights in the invention.

### REFERENCE TO A SEQUENCE LISTING SUBMITTED AS XML VIA EFS-WEB

The instant application contains a Sequence Listing that is filed electronically in .xml format and is hereby incorporated by reference in its entirety. Said .xml copy, created on Feb. 21, 2025, is named 6975-112563-03_Sequence Listing.xml and is 100,730 bytes in size.

### BACKGROUND

Alzheimer's disease (AD) is the most common form of dementia, affecting more than 45 million people worldwide. Cerebrospinal fluid (CSF) β-amyloid (Aβ), Tau, and phosphorylated Tau currently provide the most sensitive and specific biomarkers for diagnosis. However, these diagnostic biomarkers do not reflect the complex changes in AD brains beyond Aβ plaques and Tau neurofibrillary tangles (NFT) and thus fail to reflect the heterogeneous and complex changes associated with the disease. Failed clinical trials in the treatment of AD highlight the need for advancements in diagnostic profiling, disease monitoring, and treatment evaluation.

### SUMMARY

Provided herein is a sensitive, quantitative, and scalable targeted proteomics assay of AD biomarkers representing neuronal, glial, vasculature and metabolic pathways. The biomarkers are protease-digested peptides selected from biological samples of individuals having normal Aβ and Tau levels (AT−) and from symptomatic and asymptomatic individuals having low Aβ and high Tau levels (AT+). The assay uses selective reaction monitoring-based mass spectrometry (SRM-MS) of peptides in the biological samples after digestion. Isotopically labeled peptide standards are added as internal standards for relative quantification.

Thus, provided herein is a method for measuring multiple peptides indicative of cognitive function in a biological sample (e.g., a cerebrospinal fluid or plasma sample) from a subject. The method includes treating the sample from the subject with a protease to produce a peptide solution comprising multiple peptides indicative of cognitive function, wherein the multiple peptides indicative of cognitive function comprise two or more of the peptides having SEQ ID NO:1-53 and SEQ ID NO:69-116; adding to the peptide solution a reference standard comprising isotopically labeled peptides to produce a test solution; detecting the multiple peptides indicative of cognitive function and the isotopically labeled peptides in the test solution using selective reaction monitoring-based mass spectrometry; and determining an amount of the multiple peptides indicative of cognitive function. The method permits determining the Alzheimer's disease state (e.g., positive/negative, asymptomatic/symptomatic, and mild cognitive impairment/early-stage AD/late-stage AD).

Also provided herein are methods of treating a subject with or at risk of developing AD. The method comprises utilizing the method for measuring multiple peptides indicative of cognitive function in a biological sample from a subject and administering a treatment (e.g., a therapeutic agent) to the subject. Optionally the method for measuring multiple peptides indicative of cognitive function in a biological sample to detect changes in brain function and efficacy of treatment.

A kit comprising one or more reagents for performing the method of measuring multiple peptides indicative of cognitive function in a biological sample is also provided.

The details of one or more embodiments are set forth in the description below. Other features, objects, and advantages will be apparent from the description and from the claims.

### BRIEF DESCRIPTION OF THE DRAWINGS

FIGS. 1A-1F shows cohort characteristics. A total of 390 samples (133 controls, 127 asymptomatic AD (AsymAD), and 130 AD unless otherwise noted) were analyzed using the identified cohort characteristics for grouping. FIG. 1A shows the age range across each group of the cohort, which were carefully selected to balance for age and sex (see also Table 2). FIG. 1B shows the range of cognition assessed using the Montreal Cognitive Assessment (MoCA) score. There is no significant difference in scores between the Control and AsymAD groups serving as the two cognitively normal diagnostic groups; however, a significant decrease in cognition scores was observed between the controls and AsymAD and the controls and AD group (133 controls, 127 AsymAD, 124 AD). FIG. 1C shows the Roche Diagnostics Elecsys® assay platform for CSF biomarker measurements for Aβ (1-42). The concentration of Aβ (1-42) was observed to significantly decrease across the groups, with controls having the highest concentration and AD subjects displaying the lowest. FIG. 1D shows the Roche Diagnostics Elecsys® assay platform for CSF biomarker measurements for Total Tau (133 controls, 127 AsymAD, 129 AD). FIG. 1E shows the Roche Diagnostics Elecsys® assay platform for CSF biomarker measurements for phosphorylated Tau (pTau) (pg/mL). Both Total Tau and phosphorylated Tau displayed the same increase in concentration across the groups, with controls having the lowest concentration and AD subjects had the highest. FIG. 1F shows the Tau/Aβ ratio data that were used to stratify the control group from disease state using a cutoff of 0.222. 129/130 AD cases were above 0.227 Tau/amyloid thresholds, consistent with AD biomarker positivity. The significance of the pairwise comparisons is indicated by overlain annotation of ‘ns’ (not significant) or asterisks; P>0.05, ****P≤0.0001.

FIG. 2 shows the coefficients of variation (CVs) plotted for 58 biomarker peptides in AT− and AT+ control pools. The control pools were measured (n=30) during the analysis of clinical samples. The % CV for 58 biomarker peptides measured in AT−(black, n=15) and AT+ (gray, n=15) was plotted to illustrate all biomarker peptides had a CV<20%. CV=20% is shown with a dashed line.

FIGS. 3A-3D show the background peptide levels in CSF for four proteins monitored for levels of potential blood contamination in each of the CSF samples. FIG. 3A shows the peptide ratio for hemoglobin subunit alpha (HBB; SEQ ID NO: 62) plotted for each of the CSF samples (n=423) in acquisition order. FIG. 3B shows the peptide ratio for hemoglobin subunit beta (HBA1; SEQ ID NO: 61) plotted for each of the CSF samples (n=423) in acquisition order. FIG. 3C shows the peptide ratio for albumin comprising the amino acid sequence corresponding to SEQ ID NO: 59 while FIG. D shows the peptide ratio for albumin comprising the amino acid sequence corresponding to SEQ ID NO: 60 plotted for each of the CSF samples (n=423) in acquisition order.

FIG. 4 shows differentially abundant peptides representing changed proteins in AT− vs AT+ control CSF pools. The differentially abundant proteins in the control pools were used to check the accuracy of the fold change. Twenty-one (21) upregulated and 10 downregulated peptides were identified. This result validated the direction of change of six proteins nominally significantly downregulated in previously published discovery proteomics (PON1, APOC1, NPTX2, VGF, NPTXR, and SCG2), and of sixteen proteins previously reported as upregulated (YWHAZ, GDA, CHI3L1, PKM, CALM2, SMOC1, YWHAB, MDH1, ALDOA, ENO1, GOT1, PPIA, DDAH1, PEBP1, PARK7, and SPP1).

FIGS. 5A-5C show the use of isotopologue peptide internal reference standards to determine consistency of LC-MS/MS platform. Each of the CSF samples were spiked with a six-peptide, 5 isotopologue concentration LC-MS/MS Peptide Reference Mix from Promega (50 fmol/μL). FIG. 5A shows an extracted ion chromatogram for the 6 peptide (1 pmol) mixture illustrating the wide range of retention times due to their hydrophobicity. FIG. 5B shows the raw peak areas in 423 injections over 5 days used to determine the label-free coefficient of variation for each isotopologue peptide and estimating the lowest limits of detection to be between 1-10 fmole for each peptide (VTSGSTSTSR (SEQ ID NO:63); LASVSVSR (SEQ ID NO:64); YVYVADVAAK (SEQ ID NO:65); VVGGLVALR (SEQ ID NO:66); LLSLGAGEFK (SEQ ID N:67); LGFTDLFSK (SEQ ID NO:68)). FIG. 5C shows the dynamic range across the gradient profile for the six isotopolgues with each peptide demonstrating linearity across 3-4 orders of magnitude in the batch of 423 injections. Error bars represent the standard deviation across 423 injections.

FIG. 6 shows the technical reproducibility of peptide measurements in three patient case samples randomly selected from 423 SRM injections. The Pearson correlation for each replicate was measured for all 58 peptides and indicates robust reproducibility of the assay. Non-paired data for the same 58 peptides in the remaining 390×389 assay pairs were significantly (p<0.001) less correlated with a mean ρ=0.963.

FIGS. 7A-7D show differential expression analysis across stages of AD progression. ANOVA analysis with Tukey post hoc FDR was performed for pairwise comparison of mean log 2 (ratio) differences between the 3 stages of AD (i.e., Control, AsymAD, and AD) of N=390 total case samples and plotted as a volcano plots. Significance threshold for counting of peptides was (p<0.05; dashed horizontal line). FIG. 7A shows differentially expressed peptides (labeled by their gene symbols) for AsymAD (N=127) versus control (N=133). FIG. 7B shows differentially expressed peptides (labeled by their gene symbols) for AD (N=130) versus control. FIG. 7C shows differentially expressed peptides (labeled by their gene symbol) for AD versus AsymAD. FIG. 7D is a Venn diagram showing counts of peptides with significant difference in any of the 3 dichotomous comparisons.

FIGS. 8A-8B show the stratification of early and progressive AD biomarkers. FIG. 8A is a gradient heatmap showing the magnitude of positive (top) and negative (bottom) changes representing mean log 2 fold change (log 2FC) for each of the 49 peptides found significant in any of the 3 comparisons of diagnostic groups. Tukey significance of the pairwise comparisons is indicated by overlain asterisks; *p<0.05. **p<0.01. ***p<0.001. FIG. 8B shows peptide abundance levels of selected panel markers that are differentially expressed between groups. The upper row highlights early biomarkers that are significantly different in AsymAD versus controls, but not significantly different in AsymAD versus AD (GEFVTTVQQR, SEQ ID NO: 28; AQALEQAK, SEQ ID NO: 44; and QETLPSK, SEQ ID NO: 46). The middle row of 3 peptides highlights progressive biomarkers of AD, which show a stepwise increase in abundance from control to AsymAD cases and further from AsymAD to AD cases (YDNSLK, SEQ ID NO: 19; NLLSVAYK, SEQ ID NO: 51, VVSSIEQK, and SEQ ID NO: 53). The bottom row highlights an interesting set of polypeptides that are increased in AsymAD compared to control cases but decreased in AD versus control or AsymAD cases (EPVAGDAVPGPK, SEQ ID NO: 48; ELKVLQGR, SEQ ID NO: 31; and VAELEDEK, SEQ ID NO: 30), suggesting these neuronal/synaptic markers could play a role in cognitive resilience.

FIGS. 9A-9C show a receiver-operating characteristic (ROC) curve analysis of peptide diagnostic potential. ROC curves for each of three pairs of diagnosed case groups were generated to determine the top-ranked diagnostic biomarker peptides among the 58-peptide panel. FIG. 9A shows a total of 263 AD (N=130) and control (N=133) CSF case samples that were classified according to the logistic fit for each peptide's log 2 (ratio) measurements across these samples, and the top 5 ranked peptides by AUC are shown (VVSSIEQK, SEQ ID NO: 53; NLLSVAYK, SEQ ID NO: 51; AQALEQAK, SEQ ID NO: 44; VISSIEQK, SEQ ID NO: 52; YDNSLK, SEQ ID NO: 19). FIG. 9B shows the top 5 performing peptides (AQALEQAK, SEQ ID NO: 44; DHLLGVSDSGK, SEQ ID NO: 20; NLLSVAYK, SEQ ID NO: 51; VVSSIEQK, SEQ ID NO: 53; LFEELVR, SEQ ID NO: 39) for discerning AsymAD (N=127) from control (N=133) case diagnosis groups are provided with AUCs, nominating these peptides as potential markers of pre-symptomatic disease, and as cognates for low Aβ and high Tau levels (AT+) biomarker positivity. FIG. 9C shows symptomatic AD (N=130) and AsymAD (N=127) discerning peptides ranked by AUC and the top 5 ROC curves (VAELEDEK, SEQ ID NO: 30; ELDVLQGR, SEQ ID NO: 31: EPVAGDAVPGPK, SEQ ID NO: 48; IESQTQEEVR, SEQ ID NO: 43; VVSSIEQK, SEQ ID NO: 53) are shown and nominated as cognate CSF measures for compromised patient cognition.

FIGS. 10A-10F show peptide abundance levels of a selected panel of markers in Caucasians and African Americans with or without AD. FIG. 10A shows significantly increased levels of SMOC1 peptide (SEQ ID NO:44) in AD but significantly lower levels in African Americans (AD-AA) versus Caucasians (AD-Cau) with AD. FIG. 10B shows significantly lower levels of VGF peptide (SEQ ID NO:48) in African Americans as compared to Caucasians with AD. FIG. 10C similarly shows significantly lower levels of SCG2 peptide (SEQ ID NO:43) in African Americans as compared to Caucasians with AD. FIG. 10D shows significantly lower levels of PKM peptide (SEQ ID NO:38) in African Americans as compared to Caucasians with AD. FIG. 10E shows that ENO1 peptide (SEQ ID NO:15) increases proportionally in AD for both races. FIG. 10F similarly shows GAPDH peptide (SEQ ID NO:19) also increases proportionally in AD for both races. ANOVA Tukey significance of the pairwise comparisons is indicated by overlain asterisks: *p<0.05. **p<0.01. ***p<0.001.

FIG. 11 shows peptide changes after drug treatment. Examples of biomarker responses observed for peptide DHLLGVSDSGK (SEQ ID NO:20) corresponding to GDA (left), a protein linked to synaptic biology in brain, and peptide LNVTEQEK (SEQ ID NO:15) ENO1 (right), a protein linked to brain metabolism. Both proteins were decreased (ANOVA Tukey p<. 05) in CSF after treatment with atomoxetine (ATX), indicating a treatment response. The decrease in abundance of these proteins with treatment is directionally “normalizing”; i.e., levels of both proteins are increased in AD samples versus controls, with ATX treatment reducing levels to levels found in controls.

FIGS. 12A-12C show a ROC curve analysis of the optimal combination of peptides as determined by machine learning and explainable AI. ROC curves for each of the three pairs of diagnosed case groups were generated and the combination of peptides (from a 58-peptide panel) with best discriminative ability was identified. FIG. 12A shows the total of 263 AD (N=130) and control (N=133) CSF case samples classified using the peptide's log 2 (ratio) measurements across the samples, AUC of the optimal combination of peptides are shown. FIG. 12B shows the optimal combination of peptides for discerning AsymAD (N=127) from control (N=133) case diagnosis groups with AUCs, nominating these peptides as potential markers of pre-symptomatic disease state and as cognates for AT+ biomarker positivity. FIG. 12C shows symptomatic AD (N=130) and AsymAD (N=127) discerning the optimal combination of peptides with the best AUC and nominated as cognate CSF measures for compromised patient cognition.

FIGS. 13A-13C show SHAP analysis using a machine learning algorithm to arrive at a classification. In this instance, SHAP analysis reveals the relative importance of each of the peptides in the decision to classify a subject into one of the three cohorts-AD, AsymAD, Control. FIG. 13A shows the decision plot for each of the 260 subjects (AD=127, dashed lines on the right; Control=133, solid lines on the left) for peptides VVSSIEQK (SEQ ID NO:53), LGADMEDVCGR (SEQ ID NO:57), AQALEQAK (SEQ ID NO:44), IESQTQEEVR (SEQ ID NO:43), NLLSVAYK (SEQ ID NO:51), LEGNPIVLGK (SEQ ID NO:33), AGALNSNDAFVLK (SEQ ID NO:22), VAELEDEK (SEQ ID NO:30), EPVAGDAVPGPK (SEQ ID NO:48), IEEELGSK (SEQ ID NO:14), AAFNSGK (SEQ ID NO:18), ELDVLQGR (SEQ ID NO:31), LGADMEDVR (SEQ ID NO:55), IASNTQSR (SEQ ID NO:9), QETLPSK (SEQ ID NO:46), YDNSLK (SEQ ID NO:19), VSFELFADK (SEQ ID NO:41), LNVTEQEK (SEQ ID NO:15), VISSIEQK (SEQ ID NO:52), and GLQEAAEER (SEQ ID NO:49). FIGS. 13B and 13C showcase waterfall plots displaying the underlying contribution of each peptide to a predicted AD state. FIG. 13B is an example of a patient classified accurately as belonging to the Control cohort (FIG. 13B) with 9 peptides (SEQ ID NOs: 57, 43, 53, 51, 33, 30, 48, 55, and 31, shown from top) showing the highest impact. FIG. 13C shows another patient classified accurately as belonging to the AD cohort with 9 peptides (SEQ ID Nos: 53, 48, 30, 43, 57, 44, 22, 31, and 14, shown from top) showing the highest impact for this individual.

FIGS. 14A-14B show the correlation of peptide biomarker abundances to Amyloid, Tau and cognitive measures. In FIG. 14A, positive (top) and negative (bottom) Pearson correlations are shown between peptide abundance (for peptides with the amino acid sequences of SEQ ID NOs: 51, 53, 52, 44, 38, 19, 39, 18, 7, 9, 28, 20, 46, 1, 35, 15, 14, 41, 21, 16, 36, 12, 27, 45, 47, 13, 25, 26, 42, 24, 29, 61, 32, 8, 34, 56, 49, 43, 48, 31, 22, 11, 33, 30, 5, 6, 2, 37, 17, 10, 23, 3, 40, 50, 4, 59, and 60, shown from top) and ELISA measures of amyloid beta (AB) 1-42, total Tau, pTau, the ratio of total Tau/Aß, and cognition (MoCA). Student's significance is indicated by overlain asterisks; *p<0.05. **p<0.01, ***p<0.001. FIG. 14B shows individual correlation scatterplots for SMOC1 (SEQ ID NO:44) (upper row), YWHAZ (SEQ ID NO:53) (middle row), and VGF (SEQ ID NO:48) (lower row). Significant correlations of these peptides to the established biomarker and cognitive measures indicate these measurements can classify or stage disease progression. Individual cases are colored by their diagnosis; solid black circles for controls, textured circles for AsymAD cases, and solid white circles for AD cases.

### DETAILED DESCRIPTION

Proteins are the proximate mediators of disease, integrating the effects of genetic, epigenetic, and environmental factors. Network proteomic analysis has emerged as a valuable tool for organizing complex unbiased proteomic data into groups or “modules” of co-expressed proteins that reflect various biological functions. CSF and plasma samples contain proteins associated with brain functions, including functions associated with neuronal, glial, vascular, and metabolic pathways. Provided herein is an assay for detecting and measuring selected peptides that are robustly detected with good precision and differentially expressed in various AD states and stages of progression. Because AD has a characteristic pre-clinical or asymptomatic period (AsymAD) in which individuals have AD neuropathology in the absence of clinical cognitive decline, detection at the prodromal phase of AD means that disease intervention, clinical trial stratification, and monitoring drug efficacy can begin earlier than has previously been possible. Similarly, classification of various Alzheimer's Disease states can provide insight into state of progressions and effectiveness of treatment.

Thus, provided herein is a method for measuring multiple peptides indicative of cognitive function in a biological sample from a subject. The method includes treating the biological sample from the subject with an enzyme to produce a peptide solution comprising multiple peptides indicative of cognitive function. The multiple peptides indicative of cognitive function comprise two or more of the different peptides, each having an amino acid sequence of any one of SEQ ID NO:1-53 and SEQ ID NO:69-116. The method further comprises adding to the peptide solution a reference standard comprising isotopically labeled peptides to produce a test solution. Multiple peptides indicative of cognitive function and the isotopically labeled peptides are detected in the test solution using selective reaction monitoring-based mass spectrometry (SRM-MS). The method also includes determining an amount of the multiple peptides indicative of cognitive function.

The biological sample can be, for example, a CSF sample, a plasma sample, or an CSF or plasma sample enriched for one or more selected peptides. Molecules in the CSF can include neurotransmitters, peptides, and other neuroactive substances wherein the presence of any one of these molecules can serve as a biomarker for disease diagnosis, progression, and/or treatment response. To measure the concentration of any one of the above-mentioned molecules, a CSF sample can be collected (e.g., from the spinal cord via lumbar puncture using a spinal needle). Plasma is separated from a blood sample, typically acquired by venipuncture, by adding an anticoagulant to the blood sample and centrifuging at sufficient speed to separate the plasma from the blood cells.

In the methods provided herein, one or more polypeptides in either a CSF sample or a plasma sample can be detected by mass spectrometry (e.g., by SRM-MS). Alternative methods for detecting polypeptides include but are not limited to Western blot, enzyme-linked immunosorbent assay (ELISA), enzyme immunoassay (EIA), or radioimmunoassay (RIA). Concentrations for most such polypeptides that comprise the CSF or plasma proteomic network can differ as the brain is bathed in CSF.

Subject, as used herein, refers to a mammal, such as a human or non-human primate, wherein the mammalian subject can be of any age, including an adult subject. In any of the methods set forth herein, the subject can be suspected of having AD, diagnosed with AD, or at risk of developing AD. Risk factors associated with AD include demographic factors (e.g., age, gender, race and social class), genetics (e.g., amyloid precursor protein, presenilin, and Apolipoprotein E (APOE)), lifestyle (e.g., substance abuse, smoking, and sedentary lifestyle), disease (e.g., cardiovascular disease or infection), psychiatric status (e.g., depression), and environmental factors (e.g., exposure to pollutants and metals, vitamin deficiencies).

As used throughout, cognitive function describes a subject's performance in brain activities such as attention, memory, processing speed, and executive function (i.e., reasoning, planning, problem solving, and multitasking). Subjects can show signs of decline in cognitive function characterized, for example, by progressive loss of memory, cognition, reasoning, judgment, and emotional stability. Declines in cognitive function may be related to Alzheimer's disease or mild cognitive impairment (MCI), but could be due to numerous other causes such as but not limited to psychosis, stroke, traumatic brain injury, and the like.

Methods for diagnosis or assessment of a subject having cognitive function impairment or a related condition are well-known in the art and are routinely conducted by a physician or other medical professional. For example, a variety of tests known to those skilled in the art can be used to demonstrate cognitive impairment, or the lack thereof, in a human. These tests include, but are not limited to, the Alzheimer's Disease Assessment Scale-cognitive subscale (ADAS-cog), the clinical global impression of change scale (CIBIC-plus scale), the Alzheimer's Disease Cooperative Study Activities of Daily Living Scale (ADCS-ADL), the Mini Mental State Exam (MMSE); the Neuropsychiatric Inventory (NPI), the Clinical Dementia Rating Scale (CDR), the Cambridge Neuropsychological Test Automated Battery (CANTAB), and the Sandoz Clinical Assessment-Geriatric (SCAG). In addition, cognitive function may be measured using imaging techniques such as Positron Emission Tomography (PET), functional magnetic resonance imaging (fMRI), or Single Photon Emission Computed Tomography (SPECT) to measure brain activity. In animal model systems, cognitive impairment can be measured in any number of ways known in the art, including using the Morris Water Maze or Object Recognition Task.

Enzymatic treatment of the biological sample optionally comprises treatment with a one or more proteases to produce a peptide solution. Such proteases include trypsin, Lys-C, and Lys-N, which can be used alone or in combination. For example, the biological sample can be treated with a combination of Lys-C and trypsin. Enzymatic treatment produces a peptide solution comprising multiple peptides indicative of cognitive function, including those peptides having amino acid sequences SEQ ID NO:1-53 and SEQ ID NO:69-116. These peptides correspond to one or more proteins indicative of neuronal, glial, vascular, or metabolic brain functions. Multiple peptides may correspond to different peptide fragments of the same protein. The method comprises detecting at least two peptides, which can be peptides corresponding to the same or different proteins and can be peptides corresponding to proteins related to different brain functions. The multiple peptides indicative of cognitive function optionally comprises at least two, three, four, five, six, seven, eight, nine, or ten peptides selected from the group consisting of AAFNSGK (SEQ ID NO:18), AGALNSNDAFVLK (SEQ ID NO22), ALVILAK (SEQ ID NO:35), AQALEQAK (SEQ ID NO:44), DHLLGVSDSGK (SEQ ID NO:20), EAFSLFDK (SEQ ID NO:7), ELDVLQGR (SEQ ID NO:31), EPVAGDAVPGPK (SEQ ID NO:48), GLQEAAEER (SEQ ID NO:49), GQLSFNLR (SEQ ID NO:24), IASNTQSR (SEQ ID NO:9), IEEELGSK (SEQ ID NO:18), IESQTQEEVR (SEQ ID NO:43), LAIGFSTVQK (SEQ ID NO:32), LEGNPIVLGK (SEQ ID NO:33), LFEELVR (SEQ ID NO:39), LNVTEQEK (SEQ ID NO:15), NLLSVAYK (SEQ ID NO:51), QETLPSK (SEQ ID NO:46), QSELSAK (SEQ ID NO:3), VAELEDEK (SEQ ID NO:30), VISSIEQK (SEQ ID NO:52), YDNSLK (SEQ ID NO:19), and VVSSIEQK (SEQ ID NO:53). For example, the peptides can include VISSIEQK (SEQ ID NO:52), VVSSIEQK (SEQ ID NO:53), and NLLSVAYK (SEQ ID NO:51). Optionally, the tested peptides include peptides indicative of APOE expression including, for example, one or more of CLAVYQAGAR (SEQ ID NO:54) specific for APOE2, LGADMEDVR (SEQ ID NO:55) specific for APOE4, ELQAAQAR (SEQ ID NO: 56) specific for APOE, LGADMEDVCGR (SEQ ID NO:57) specific for APOE2 or APOE3, and LAVYQAGAR (SEQ ID NO:54) specific for APOE3 or APOE4. More specifically the peptides tested can include LGADMEDVCGR (SEQ ID NO:57) and LGADMEDVR (SEQ ID NO:55). Optionally, the multiple peptides indicative of cognitive function comprise at least two, three, four, five, six, seven, eight, nine, or ten peptides selected from the group consisting of AAQEEYVK (SEQ ID NO: 69), ADQDTIR (SEQ ID NO: 70), DGADFAK (SEQ ID NO: 71), DGNGYISAAELR (SEQ ID NO: 72), DIEEGAIVNPGR (SEQ ID NO: 73), DYSVTANSK (SEQ ID NO: 74), EGDCPVQSGK (SEQ ID NO: 75), EHAVEGDCDFQLLK (SEQ ID NO: 76), ELSDIAHR (SEQ ID NO: 77), ENFSCLTR (SEQ ID NO: 78), EPCGGLEDAVNEAK (SEQ ID NO: 79), ESLSSYWESAK (SEQ ID NO: 80), EVTGIITQGAR (SEQ ID NO: 81), FIVYSYK (SEQ ID NO: 82), FVEGLPINDFSR (SEQ ID NO: 83), GALQNIIPASTGAAK (SEQ ID NO: 84), GDLGIEIPAEK (SEQ ID NO: 85), GDSVVYGLR (SEQ ID NO: 86), GDYPLEAVR (SEQ ID NO: 87), GECVPGEQEPEPILIPR (SEQ ID NO: 88), GNDISSGTVLSDYVGSGPPK (SEQ ID NO: 89), GNQWVGYDDQESVK (SEQ ID NO: 90), GVCEETSGAYEK (SEQ ID NO: 91), GVNLPGAAVDLPAVSEK (SEQ ID NO: 92), HVLFGTVGVPEHTYR (SEQ ID NO: 93), HYGGLTGLNK (SEQ ID NO: 94), ICEPGYSPTYK (SEQ ID NO: 95), IVFLEEASQQEK (SEQ ID NO: 96), LIVHNGYCDGR (SEQ ID NO: 97), LLVFATDDGFHFAGDGK (SEQ ID NO: 98), LYEQLSGK (SEQ ID NO: 99), SGQLGIQEEDLR (SEQ ID NO: 100), TATSEYQTFFNPR (SEQ ID NO: 101), TEAADLCK (SEQ ID NO: 102), TLLSVGGWNFGSQR (SEQ ID NO: 103), VFEDESGK (SEQ ID NO: 104), VGNLTVVGK (SEQ ID NO: 105), VIGSGCNLDSAR (SEQ ID NO: 106), VIVVGNPANTNCLTASK (SEQ ID NO: 107), VNQIGSVTEAIQACK (SEQ ID NO: 108), VTLSAAPPSYFR (SEQ ID NO: 109), VVEGSFVYK (SEQ ID NO: 110), WGLGGTCVNVGCIPK (SEQ ID NO: 111), YGFIEGHVVIPR (SEQ ID NO: 112), YISPDQLADLYK (SEQ ID NO: 113), YLAEVATGEK (SEQ ID NO: 114), YLIPNATQPESK (SEQ ID NO: 115), and YVWLVYEQDRPLK (SEQ ID NO: 116).

An internal reference standard comprising, for example, isotopically labeled peptides, is added to the peptide solution to create the test solution and the amount of each multiple peptide indicative of cognitive function is determined relative to the internal standard. The isotopically labeled peptides optionally comprise peptides having the amino acid sequences of SEQ ID NO:63-68. Each isotopically labeled peptide optionally comprises a C-terminal lysine or arginine residues labeled with ¹³C, ¹⁵N or both ¹³C and ¹⁵N. During liquid chromatography, the mass altered peptide will elute at the same location as its corresponding non-mass altered peptide, thus serving as an internal standard that allows for absolute quantification of the amount of peptide in a sample.

Detection of the multiple peptides in the test solution optionally is by a selective reaction monitoring-based mass spectrometry (SRM-MS) method. SRM-MS, also referred to as SRM herein, is a method for detecting and quantifying specific, predetermined analytes (e.g., metabolites, drugs, peptides, and the like) with known fragmentation properties. The SRM step comprises a targeted liquid chromatography-tandem mass spectrometry method. In some of the methods provided herein, a known concentration of isotopically labeled peptide standards are added, or spiked, into the peptide solution and used for relative quantification of the one or more targeted peptides. The ratio of internal standard (e.g., isotopically labeled peptides) to the one or more target peptides is determined by comparing the SRM results of the target peptides with a standard curve generated from the SRM analysis. This ratio can be further used to determine the amount of peptide in the sample.

In the methods provided herein, mass spectrometry peak volume can be calculated by detecting and determining peak shape for a given mass during elution from an LC-MS system. Since the isotopically labeled peptides have known masses and the one or more target peptides have known masses, the intensity of the peaks corresponding to these masses can be tracked during the elution period. Numerous software programs are available for detecting and determining the intensity of these peaks, for example, Skyline-daily software available from Altis TSQ.

Based on the results of the assay method, the amounts of the selected peptides indicative of cognitive function can be used to identify an AD state in the subject. As used herein, an AD state refers to distinguishing a general AD state of positive versus negative, a clinical AD state of prodromal (i.e., asymptomatic) versus symptomatic, or to reflect a stage such as mild cognitive impairment, early-stage AD, versus late-stage AD. Both asymptomatic and symptomatic AD subjects display AD neuropathology; however, asymptomatic individuals do not show symptoms of cognitive function decline. Subjects presenting with mild cognitive impairment may be at risk for developing AD. Optionally, the Alzheimer's Disease state is further characterized as low Aβ and high Tau levels (AT+) or normal Aβ and Tau levels (AT−). These distinctions in AD state can be identified based on selected peptides in a sample from the subject.

By way of example, one or more of the following peptide sequences shown in Table 1 can be used to distinguish AD versus control, AD vs. asymptomatic AD, and Asymptomatic AD versus control. In some cases, the peptide level is elevated in AD as compared to control and in some cases the peptide level is reduced in AD as compared to control.

TABLE 1

| AD vs. Control | AD vs. AsymAD | AsymAD vs. Control |
|---|---|---|
| AAFNSGK | AAFNSGK | AQALEQAK |
| SEQ ID NO: 18 | SEQ ID NO: 18 | SEQ ID NO: 44 |
| AGALNSNDAFVLK | ELDVLQGR | DHLLGVSDSGK |
| SEQ ID NO: 22 | SEQ ID NO: 31 | SEQ ID NO: 20 |
| ALVILAK | EPVAGDAVPGPK | IEEELGSK |
| SEQ ID NO: 35 | SEQ ID NO: 48 | SEQ ID NO: 14 |
| AQALEQAK | GLQEAAEER | LFEEL VR |
| SEQ ID NO: 44 | SEQ ID NO: 49 | SEQ ID NO: 39 |
| DHLLGVSDSGK | GQLSFNLR | LGADMEDVCGR |
| SEQ ID NO: 20 | SEQ ID NO: 24 | SEQ ID NO: 57 |
| EAFSLFDK | IESQTQEEVR | LGADMEDVR |
| SEQ ID NO: 7 | SEQ ID NO: 43 | SEQ ID NO: 55 |
| ELDVLQGR | LAIGFSTVQK | QSELSAK |
| SEQ ID NO: 31 | SEQ ID NO: 32 | SEQ ID NO: 3 |
| EPVAGDAVPGPK | VAELEDEK | VISSIEQK |
| SEQ ID NO: 48 | SEQ ID NO: 30 | SEQ ID NO: 52 |
| GLQEAAEER | VISSIEQK | VSFELFADK |
| SEQ ID NO: 49 | SEQ ID NO: 52 | SEQ ID NO: 41 |
| IASNTQSR | VVSSIEQK | VVSSIEQK |
| SEQ ID NO: 9 | SEQ ID NO: 53 | SEQ ID NO: 53 |
| IEEELGSK | YDNSLK | NLLSVAYK |
| SEQ ID NO: 14 | SEQ ID NO: 19 | SEQ ID NO: 51 |
| IESQTQEEVR | NLLSVAYK | |
| SEQ ID NO: 43 | SEQ ID NO: 51 | |
| LEGNPIVLGK | | |
| SEQ ID NO: 33 | | |
| LFEELVR | | |
| SEQ ID NO: 39 | | |
| LGADMEDVCGR | | |
| SEQ ID NO: 57 | | |
| LGADMEDVR | | |
| SEQ ID NO: 55 | | |
| LNVTEQEK | | |
| SEQ ID NO: 15 | | |
| NLLSVAYK | | |
| SEQ ID NO: 51 | | |
| QETLPSK | | |
| SEQ ID NO: 46 | | |
| VAELEDEK | | |
| SEQ ID NO: 30 | | |
| VISSIEQK | | |
| SEQ ID NO: 52 | | |
| VSFELFADK | | |
| SEQ ID NO: 41 | | |
| VVEVGSK | | |
| SEQ ID NO: 38 | | |
| VVSSIEQK | | |
| SEQ ID NO: 53 | | |
| YDNSLK | | |
| SEQ ID NO: 19 | | |

The multiple peptides indicative that a subject is Alzheimer's Disease positive optionally comprise peptide fragments of glucose metabolism enzyme genes such as, but not limiting to PKM, MDH1, ENO1, ALDOA, ENO2, LDHB, and TPI1. Glucose metabolism and the enzymes that function in this pathway work to breakdown complex carbohydrate molecules into simple sugars such as glucose, fructose, mannose, and galactose, that are released into the blood stream and used for energy. Glucose is the sole source of energy for the brain, thus alterations to glucose metabolism that cause reductions in blood glucose have a profound impact on brain health and contribute to AD and its progression. Additional peptides indicative of being Alzheimer's Disease positive can further comprise having two or more peptides having the amino acid sequence of SEQ ID NO:44, SEQ ID NO:20, SEQ ID NO:39, SEQ ID NO:57, SEQ ID NO:55, SEQ ID NO:51, SEQ ID NO:53 SEQ ID NO:52, SEQ ID NO:96, SEQ ID NO:115, or SEQ ID NO:19.

Multiple peptides indicative of the asymptomatic AD state optionally comprise at least two or more peptides having the amino acid sequence of SEQ ID NO:44, SEQ ID NO:55, SEQ ID NO:20, SEQ ID NO:51, SEQ ID NO:96, SEQ ID NO:115 or SEQ ID NO:53. On the other hand, peptides indicative of symptomatic AD may comprise at least two peptides from the group containing SEQ ID NO:30, SEQ ID NO:31, SEQ ID NO:48, SEQ ID NO:43, SEQ ID NO:70, SEQ ID NO:100 or SEQ ID NO:53.

One of the advantages of the present method is that using the SRM-MS method described herein permits concurrent genotyping of the subject as one or more peptides indicative of APOE, ALB, HBA, or HBB expression can be detected in the test solution.

This method can further comprise detecting the one or more peptide fragments of apolipoprotein E (apoE), albumin, hemoglobin subunit A, or hemoglobin subunit B concurrently by SRM-MS. By way of example, APOE has three major genetic variants (E2, E3, and E4, encoded by the ε2, ε3 and ε4 alleles, respectively). The variants differ by a single amino acid substitution. APOE genotype is closely related to AD risk with apoE4 having the highest risk, apoE2 the lowest risk, and apoE3 with intermediate risk. Thus, allele specific peptides can be targeted by the present SRM-MS method, for example, by detecting one or more peptides having amino acids sequences of SEQ ID NO:54-58 to detect expression of APOE2, APOE3, or APOE4. By way of example, the genotyping peptides can be CLAVYQAGAR (SEQ ID NO:54) specific for APOE2, LGADMEDVR (SEQ ID NO:55) specific for APOE4, ELQAAQAR (SEQ ID NO: 56) specific for APOE, LGADMEDVCGR (SEQ ID NO:57) specific for APOE2 or APOE3, and LAVYQAGAR (SEQ ID NO:54) specific for APOE3 or APOE4.

The dataset generated by the methods described herein can be optimized for each individual by selecting the most accurate peptides from the multiple peptides indicative of cognitive function. By way of example, selection for the most accurate peptides among those having the amino acids of SEQ ID NO:1-53 and SEQ ID NO:69-116 for a given individual can be determined using Shapley Additive explanations (SHAP). In some of the methods described herein, SHAP analysis is used to explain the output of any machine learning algorithm, wherein the output may be a classification of a subject into one of the three cohorts-AD, AsymAD, or Control. Further, the SHAP values represent the contribution or importance of each feature included in a machine learning algorithm. For example, the relative importance of each of the peptides in the decision to classify a subject as AD. Thus, the skilled artisan can optimize interpretation of the results for each subject as shown in the Examples.

Additionally, datasets can be used to eliminate racial bias in testing. By way of example, the amount of the multiple peptides indicative of cognitive function can be interpreted to correct for racial differences in expression of selected peptides. For example, one or more of peptide fragments of SMOC1, PKM, VGF, SCG1, or SCG2 can be viewed differently based on whether the subject is African American or Caucasian. More specifically, peptides measuring SMOC1 and PKM are increased in AD in both African Americans and Caucasians, SMOC1 and PKM levels are significantly lower in African Americans with AD compared to Caucasian with AD. In contrast, peptides quantifying neuronal markers VGF and SCg1 are decreased in AD in both races, but levels of VGF and SCG2 are significantly lower in African Americans with AD compared to Caucasians. Other peptides indicative of brain function (e.g., ENO1, and GAPDH) are increased proportionally in both African and Caucasian populations and do not diverge by race. Identification of such differences permits the skilled artisan to interpret the results of the present method without racial bias.

Also provided herein are methods of treating a subject with or at risk of developing AD. The treatment method includes performing the SRM-MS method described herein and selecting and administering treatment based on the results of method. Such treatment can be provided in a symptomatic or asymptomatic subject. Optionally, the SRM-MS method is repeated after treatment to track progression or improvement based on therapeutic intervention. Treatment refers to improving or slowing progression of one or more symptoms of AD in the subject being treated. Treatment can include providing to the subject an effective amount of a therapeutic agent such as a biologic (e.g., aducanumab), an N-methyl D-aspartate (NMDA) antagonist (e.g., memantine), a cholinesterase inhibitor (e.g., donepezil, rivastigmine, galantamine). Treatment can also include agents for treatment of underlying pathologies such as cardiovascular disease or diabetes.

The term effective amount, as used throughout, is defined as any amount necessary to produce a desired physiologic response, for example, reducing or delaying one or more effects or symptoms of a disease or disorder. Effective amounts and schedules for administering the therapeutic agent can be determined empirically, making such determinations within the skill of one in the art. The dosage ranges for administration are those large enough to produce the desired effect in which one or more symptoms of the disease or disorder are affected (e.g., reduced or delayed). The dosage should not be so large as to cause substantial adverse side effects, such as unwanted cross-reactions, unwanted cell death, and the like. Generally, the dosage will vary with the species, age, body weight, general health, sex and diet of the subject, the mode and time of administration and severity of the particular condition and can be determined by one of skill in the art. The dosage can be adjusted by the individual physician in the event of any contraindications. Dosages can vary and can be administered in one or more doses.

The therapeutic agent described herein are administered in a number of ways depending on whether local or systemic treatment is desired. The compositions are administered via any of several routes of administration, including intraparenchymal injection, intravenously, intrathecally, intramuscularly, intracisternally, transdermally, or a combination thereof. Effective doses for any of the administration methods described herein can be extrapolated from dose-response curves derived from in vitro or animal model test systems.

Also provided herein is a kit comprising one or more reagents used in the present SRM-MS methods. For example, the kit can comprise a mixture of isotopically labeled peptides comprising peptides having the amino acid sequences of SEQ ID NO:63-68 with labeled C-terminal lysine or arginine residues. Additionally, the kit can comprise a protease (e.g., trypsin) and/or other reagents for sample preparation as described in the examples. The kit can further comprise containers for the one or more reagents.

As used herein, the term peptide, polypeptide, protein or peptide portion is used broadly herein to mean two or more amino acids linked by a peptide bond. Protein, peptide and polypeptide are also used herein interchangeably to refer to amino acid sequences unless otherwise indicated. For example, following trypsin treatment of proteins present in a biological sample, the sample contains peptides produced by trypsinization. It should be recognized that the term peptide is not used herein to suggest a particular size or number of amino acids comprising the molecule.

Optional or optionally means that the subsequently described event or circumstance can or cannot occur, and that the description includes instances where the event or circumstance occurs and instances where it does not. For example, the phrase optionally the composition can comprise a combination means that the composition may comprise a combination of different molecules or may not include a combination such that the description includes both the combination and the absence of the combination (i.e., individual members of the combination).

Publications cited herein and the material for which they are cited are hereby specifically incorporated by reference.

The examples below are intended to further illustrate certain aspects of the methods and compositions described herein and are not intended to limit the scope of the claims.

### EXAMPLES

### Example 1

### Reagents and Materials

Heavy labeled PEPotec Grade 2 crude peptides, trypsin, mass spectrometry grade, trifluoroacetic acid (TFA), foil heat seals (AB-0757), and low-profile square storage plates (AB-1127) were purchased from ThermoFisher Scientific (Waltham, MA). Lysyl endopeptidase (Lys-C), mass spectrometry grade was bought from Wako (Japan); sodium deoxycholate, CAA (chloroacetamide), TCEP (tris-2(-carboxyethyl)-phosphine), and triethylammonium hydrogen carbonate buffer (TEAB) (1 M, pH 8.5) were obtained from Sigma (St. Louis, MO). Formic acid (FA), 0.1% FA in acetonitrile, 0.1% FA in water, methanol, and sample preparation V-bottom plates (Greiner Bio-One 96-well Polypropylene Microplates; 651261) are from Fisher Scientific (Pittsburgh, PA). Oasis PRIME HLB 96-well, 30 mg sorbent per well, solid phase extraction (SPE) cleanup plates were from Waters Corporation (Milford, MA).

### CSF Collection and Immunoassay Measurements

CSF was collected by lumbar puncture and banked according to 2014 ADC/NIA best practices guidelines https://www.alz.washington.edu/BiospecimenTaskForce.html. CSF samples from all participants were collected in a standardized fashion applying common preanalytical methods. Twenty participants were asked to fast for at least 6 hours prior to lumbar puncture (LP) procedures and CSF collection. LPs were performed using a 24 g atraumatic Sprotte spinal needle (Pajunk Medical Systems, Norcross, GA) with aspiration and, after clearing any blood contamination, CSF was transferred from the syringe to 15 mL polypropylene tubes (Corning, Glendale, AZ), which were inverted several times. The CSF (0.5 mL) was aliquoted without further handling into 0.9 mL FluidX tubes (Azenta, Chemsford, MA) and placed into a dry ice/methanol bath prior to transfer to −80° C. freezers. Time from initial collection to storage at −80° C. was less than 60 minutes. Aβ(1-42), total Tau, and pTau assays were performed on CSF samples following a single freeze-thaw cycle on a Roche Cobas e601 analyzer using the Elecsys assay platform21. All assays were performed in a single laboratory following manufacturer's recommended protocols.

### Pooled CSF as Quality Controls

Two pools of CSF were generated based on Aβ(1-42), total Tau, and pTau181 levels to create AD-positive (AT+) and AD-negative (AT−) quality control standards. Each pool consisted of approximately 50 mL of CSF by pooling equal volumes of CSF from well characterized samples (˜45 unique individuals per pool) from the Emory Goizueta Alzheimer's Disease Research Center and Emory Healthy Brain Study. AD biomarker status for individual cases was determined on the Elecsys® (Roche Diagnostics, Indianapolis, IN) platform; the average CSF biomarker value is reported in parentheses. The control CSF pool (AT−) was comprised of cases with relatively high levels of Aß(1-42) (1457.3 pg/mL) and low total Tau (172.0 pg/mL) and pTau181 (15.1 pg/mL). In contrast, the AD pool (AT+) was comprised of cases with low levels of Aß(1-42) (482.6 pg/mL) and high total Tau (341.3 pg/mL) and pTau181 (33.1 pg/mL). The quality control (QC) pools were processed and analyzed identically to the CSF clinical samples reported.

### Clinical Characteristics of the Cohort

Human cerebrospinal fluid (CSF) samples from 390 individuals including 133 healthy controls, 130 patients with symptomatic AD, and 127 patients asymptomatic AD (cognitively normal but AD biomarker positive) were obtained from Emory's Goizueta Alzheimer's Disease Research Center (ADRC). All symptomatic individuals were diagnosed by expert clinicians in the ADRC and Emory Cognitive Neurology, who are subspecialty trained in Cognitive and Behavioral Neurology, following extensive clinical evaluations including detailed cognitive testing, neuroimaging, and laboratory studies. CSF samples were selected to balance for age and sex (Table 2).

TABLE 2
Cohort Characteristics

| | Sample Group | | |
|---|---|---|---|
| | CT | AsymAD | AD |
| | Sample Size | | |
| Characteristics | N = 133 | N = 127 | N = 130 |
| Sex | 99 F, 34 M | 94 F, 33 M | 97 F, 33 M |
| Age ^{a} | 66 ± 6 | 66 ± 6 | 66 ± 6 |
| MoCA ^{b} | 26.8 ± 2.0 | 26.4 ± 2.6 | 17.4 ± 5.5 |
| Aβ₄₂ ^{b} | 1394.0 ± 280.7 | 752.0 ± 238.2 | 587.0 ± 236.8 |
| tTau ^{b} | 165.8 ± 35.0 | 260.8 ± 97.2 | 375.7 ± 139.6 |
| pTau ^{b} | 14.7 ± 3.2 | 25.2 ± 11.1 | 38.5 ± 15.5 |

^{a} Age in years. Values given as average ± standard deviation
^{b} MoCA, Aβ₄₂ tTau, and pTau in pg/mL. Values given in average ± standard deviation
Abbreviations: CT, Control;
AD, Alzheimer's disease;
MoCA, Montreal Cognitive Assessment

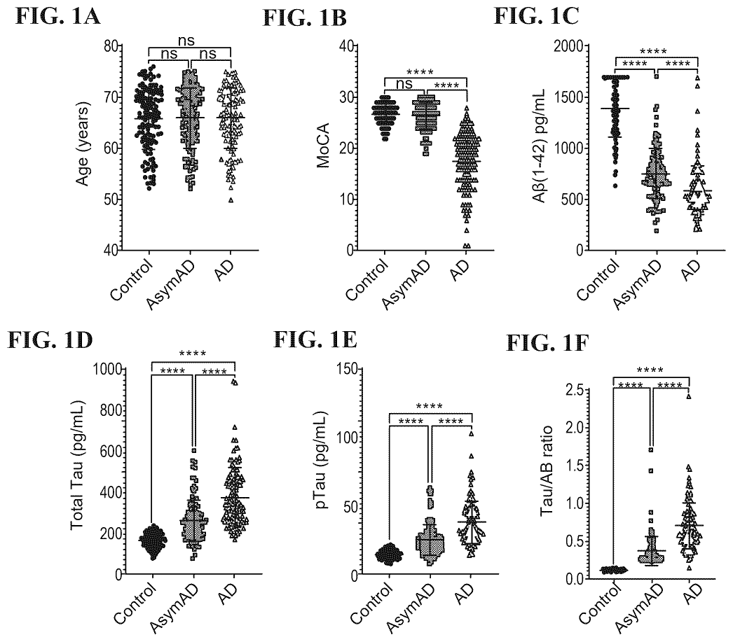

For biomarker measurements, CSF samples from all individuals were assayed for Aß42, total Tau, and pTau using the Roche Diagnostics Elecsys® IL-6 assay platform. The cohort characteristics are summarized in FIGS. 1A-1F and Table 2. Samples were stratified into controls, AsymAD and AD based on Tau and Amyloid biomarkers status and cognitive score (MoCA).

### Peptide Selection and Select Reaction Monitoring Assay

Both deep discovery and single-shot tandem mass tag (ssTMT) peptide data from CSF proteomics were used. Peptides were prioritized for SRM validation that had one or more spectral match, were differentially abundant (AD versus control), or that mapped to proteins within brain-based biological panels that differed in AD. More than 200 peptides were robustly detected and differentially expressed in CSF discovery proteomics for synthesis as crude heavy standards. The heavy crude peptides contained isotopically labeled C-terminal lysine or arginine residues (¹³C, ¹⁵N) for each tryptic peptide. Based on the crude heavy peptide signal, the peptides were pooled to achieve total area signals ≥1×10⁵ in CSF matrix. The transition lists were created in Skyline-daily software (version 21.2.1.455) (Herndon, VA). An in-house spectral library was created in Skyline based on tandem mass spectra from CSF samples. Skyline parameters were specified as trypsin enzyme, Swiss-Prot background proteome, and carbamidomethylation of cysteine residues (+57.02146 Da) as fixed modifications. Isotope modifications included ¹³C(6)¹⁵N(4) (C-term R) and ¹³C(6)¹⁵N(2) (C-term K). The top ten fragment ions that match the criteria (precursor charges: 2; ion charges 1, 2; ion types: y, b; product ion selection from m/z>precursor to last ion-2) were selected for scrutiny. The top 5-7 transitions per heavy precursor were selected by manual inspection of the data in Skyline and scheduled transition lists were created for collision energy optimization. Collision energies were optimized for each transition; the collision energy was ramped around the predicted value in 3 steps on both sides, in 2V increments. The selected transitions were tested in real matrix spiked with the heavy peptide mixtures. The three best transitions per precursor were selected by manual inspection of the data in Skyline and one scheduled transition list was created for the final assays.

### Preparation of CSF for Mass Spectrometry Analysis

All CSF samples were blinded and randomized. Each CSF sample was thawed and aliquoted into sample preparation V-bottom plates that also included quality controls. Each sample and quality control were processed independently in parallel. Crude CSF (50 μL) was reduced, alkylated, and denatured with tris-2 (-carboxyethyl)-phosphine (5 mM), chloroacetamide (40 mM), and sodium deoxycholate (1%) in triethylammonium bicarbonate buffer (100 mM) in a final volume of 150 μL. Sample plates were heated at 95° C. for 10 min, followed by a 10-min cool down at room temperature while shaking on an orbital shaker (300 rpm). CSF proteins were digested with Lys-C(Wako, Mountain View, CA: 0.5 ug; 1:100 enzyme to CSF volume) and trypsin (Pierce/ThermoFisher, Waltham, MA: 5 ug; 1:10 enzyme to CSF volume) overnight in a 37° C. oven. After digestion, heavy labeled standards for relative quantification (15 μL per 50 μL CSF) were added to the peptide solutions followed by acidification to a final concentration of 0.1% TFA and 1% FA (pH≤2). Sample plates were placed on an orbital shaker (300 rpm) for at least 10 minutes to ensure proper mixing. Plates were centrifuged (4680 rpm) for 30 minutes to pellet the precipitated surfactant. Peptides were desalted with Oasis PRIME HLB 96-well, 30 mg sorbent per well, solid phase extraction (SPE) cleanup plates from Waters Corporation (Milford, MA) using a positive pressure system. Each SPE well was conditioned (500 μL methanol) and equilibrated twice (500 μL 0.1% TFA) before 500 μL 0.1% TFA and supernatant were added. Each well was washed twice (500 μL 0.1% TFA) and eluted twice (100 μL 50% acetonitrile/0.1% formic acid). All eluates were dried under centrifugal vacuum and reconstituted in 50 μL mobile phase A (0.1% FA in water) containing Promega 6×5 LC-MS/MS Peptide Reference Mix (50 fmol/μL; Promega V7491) (Promega, Madison, WI).

### Liquid Chromatography-Tandem Mass Spectrometry (LC-MS/MS)

Peptides were analyzed using a TSQ Altis Triple Quadrupole mass spectrometer (Thermo Fisher Scientific). Each sample was injected (20 μL) using a 1290 Infinity II system (Agilent Technologies, Santa Clara, CA) and separated on an AdvanceBio Peptide Map Guard column (2.1×5 mm, 2.7 μm, Agilent) connected to AdvanceBio Peptide Mapping analytical column (2.1×150 mm, 2.7 μm, Agilent). Sample elution was performed over a 14-min gradient using mobile phase A (MPA; 0.1% FA in water) and mobile phase B (MPB; 0.1% FA in acetonitrile) with flow rate at 0.4 mL/min. The gradient was from 2% to 24% MPB over 12.1 minutes, then from 24% to 80% over 0.2 min and held at 80% B for 0.7 min. The mass spectrometer was set to acquire data in positive-ion mode using single reaction monitoring (SRM) acquisition. Positive ion spray voltage was set to 3500 V for the Heated ESI source. The ion transfer tube and vaporizer temperatures were set to 325° C. and 375° C., respectively. SRM transitions were acquired at Q1 resolution 0.7 FWHM, Q2 resolution 1.2 FWHM, CID gas 1.5 mTorr, 0.8 s cycle time.

### Data Analysis

Raw files from Altis TSQ were uploaded to Skyline-daily software (version 21.2.1.455), which was used for peak integration and quantification by peptide ratios. SRM data were manually evaluated in Skyline by assessing retention time reproducibility, matching light and heavy transitions using Ratio Dot Product, and determining the peptide ratio precision using coefficient of variation (CV) by QC condition. If Skyline could not automatically pick a consistent peak due to interference in the light transitions the peptide was removed from the analysis. Transition profiles were checked to insure the heavy and light transition profiles matched using the Ratio Dot Product value in Skyline. The Ratio Dot Product (1=exact match) is a measure of whether the transition peak areas in the two label types are in the same ratio to each other. The average Ratio Dot Product value for each peptide was >0.90 for each QC. If the retention time or Ratio Dot Product were outside of the expected range for a peptide in a few samples, the peaks were checked individually and adjusted as necessary. Total area ratios for each peptide were calculated in Skyline by summing the area for each light (3) and heavy (3) transition and dividing the light total area by the heavy total area. The Total Area Ratio CV was assessed using Skyline and the peptide was removed from the analysis if the CV>20% by QC condition. Next, the individual CSF samples were analyzed in a blinded fashion. Total area ratios for each target peptide were calculated in Skyline by summing the area for each light (3) and heavy (3) transition and dividing the light total area by the heavy total area. The total area ratios (peptide ratios) for each targeted peptide in each sample and QC analysis were used. The Data Matrix is a table of peptide ratios without imputation. The data matrix does not contain blank cells or missing data; however, there were zero measures for the APOE2 allele-specific peptide because it was not present in those samples (reviewed manually) due to genetic background.

### Statistical Analyses

Skyline-daily software (version 21.2.1.455) and GraphPad Prism (version 9.4.1) software (GraphPad Prism, San Diego) were used to calculate means, medians, standard deviations, and coefficients of variations. Peptide abundance ratios were log 2-transformed and zero values were imputed as one-half the minimum nonzero abundance measurement. Then, one-way ANOVA with Tukey post hoc tests for significance of the paired groupwise differences across diagnosis groups was performed in R using a custom calculation and volcano plotting framework implemented and available as an open-source set of R functions documented further on https://www.github.com/edammer/parANOVA. T test p values and Benjamini-Hochberg FDR for these are reported for two total group comparisons, as was the case for AT+ versus AT− peptide mean difference significance calculations. ROC analysis was performed in R version 4.0.2 with a generalized linear model binomial fit of each set of peptide ratio measurements to the binary case diagnosis subsets AD/Control, AsymAD/Control, and AD/AsymAD using the pROC package implementing ROC curve plots, and calculations of AUC and AUC DeLong 95% confidence interval. Additional ROC curve characteristics including sensitivity, specificity, and accuracy were calculated with the reportROC R package. Robustness of the ROC calculations of AUC were confirmed using k-fold cross-validation (k=10 folds, with each fold containing case subsets with equal distributions of the binary outcome) implemented using the cvAUC R package functions for calculating cross-validated AUC (cvAUC), and confidence interval on pooled predictions, and these calculations were consistently within 1 percent of AUC as calculated using a single calculation on the full data. Venn diagramming was performed using the R vennEuler package, and the heatmap was produced using the R pheatmap package/function. R boxplot function output was overlaid with beeswarm-positioned individual measurement points using the R beeswarm package. Pearson correlations of SRM peptide measurements to immunoassay measurements of Aβ(1-42), total Tau, phospho-T181 Tau, and the ratio of total Tau/Aβ were performed using the corAndPvalue WGCNA function in R. Correlation scatterplots were generated using the verboseScatterplot WGCNA function.

### Assessing Peptide Precision Using Pooled CSF Quality Control (QC) Standards

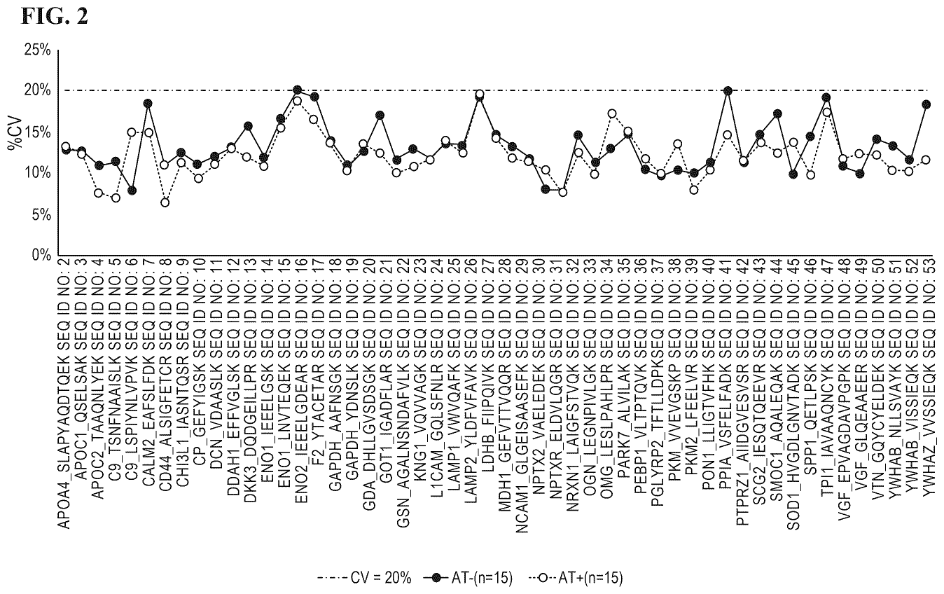

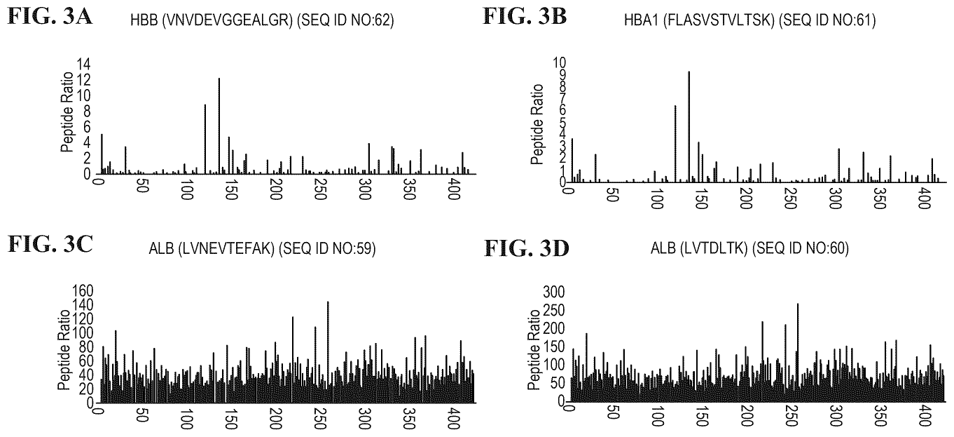

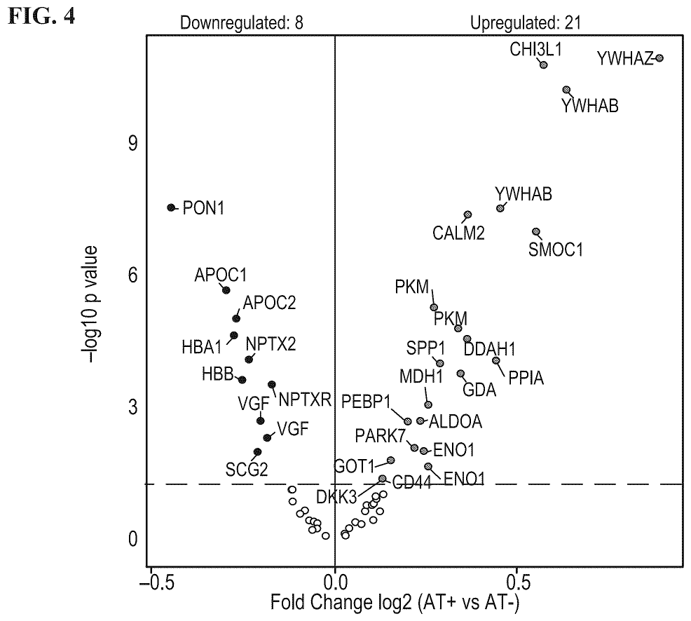

Two pools of CSF reference standards were generated as QCs based on biomarker status (AT− and AT+). These QCs were processed and analyzed (at the beginning, end, and after every 20 samples per plate) identically to the individual clinical samples for testing assay reproducibility. Thirty (30) QCs (15 AT− and 15 AT+) were evaluated over approximately 5 days during the run of clinical samples. Sixty-two (62) peptides from 51 proteins were reliably measured in the pooled reference standards. APOE (5 peptides), ALB (2 peptides), HBA (1 peptide), and HBB (1 peptide) peptides were used to determine the genotype and to monitor as background peptides. Fifty-eight (58) peptides from 51 proteins were included in the biomarker analysis, excluding the four APOE allele specific peptides. The technical coefficient of variation (CV) of each peptide was calculated based on the peptide area ratio for the biomarker negative (AT−) and positive (AT+) QCs. CSF peptide biomarkers with CVs≤20% were defined and quantified with high precision in these technical replicates, which were un-depleted and unfractionated CSF sample pools. Technical and process reproducibility for all reported peptides was below 20% (CV<20%) in at least one pooled reference standard (FIG. 2). Table 3 contains the QC statistics for the biomarker, background, and APOE allele specific peptides. Table 4 comprises additional peptides, without being limiting, that serve as biomarkers for AD. Levels of HBA, HBB and ALB peptides can be used to assess the levels of potential blood contamination in each of the CSF samples across individual plates (FIGS. 3A-3D). We used the protein directions of change to assess accuracy in the QC pools. The volcano plot between 54 peptides measured in the pools highlights peptide/protein levels that are consistent with previously reported AD biomarkers (FIG. 4).

TABLE 3

| | | | | | | |
|---|---|---|---|---|---|---|
| | Protein | | | | | |
| Protein | Accession | | | | | |
| Gene | Number | Protein Name | Peptide | AT− | AT+ | SEQ ID NO |

Biomarker Peptides of Interest

| | | | | | | |
|---|---|---|---|---|---|---|
| ALDOA | P04075 | ALDOA | VLAAVYK | 13% | 13% | 1 |
| APOA4 | P06727 | APOA4 | SLAPYAQDTQEK | 13% | 12% | 2 |
| APOC1 | P02654 | APOC1 | QSELSAK | 11% | 8% | 3 |
| APOC2 | P02655 | APOC2 | TAAQNLYEK | 11% | 7% | 4 |
| C9 | P02748 | CO9 | TSNFNAAISLK | 8% | 15% | 5 |
| C9 | P02748 | CO9 | LSPIYNL VPVK | 18% | 15% | 6 |
| CALM2 | P0DP24 | CALM2 | EAFSLFDK | 11% | 7% | 7 |
| CD44 | P16070 | CD44 | ALSIGFETCR | 13% | 11% | 8 |
| CHI3L1 | P36222 | CH3L1 | IASNTQSR | 11% | 9% | 9 |
| CP | P00450 | CERU | GEFYIGSK | 12% | 11% | 10 |
| DCN | P07585 | PGS2 | VDAASLK | 13% | 13% | 11 |
| DDAH1 | O94760 | DDAH1 | EFFVGLSK | 16% | 12% | 12 |
| DKK3 | Q9UBP4 | DKK3 | DQDGEILLPR | 12% | 11% | 13 |
| ENO1 | P06733 | ENOA | IEEELGSK | 17% | 16% | 14 |
| ENO1 | P06733 | ENOA | LNVTEQEK | 20% | 19% | 15 |
| ENO2 | P09104 | ENOG | IEEELGDEAR | 19% | 17% | 16 |
| F2 | P00734 | THRB | YTACETAR | 14% | 14% | 17 |
| GAPDH | P04406 | G3P | AAFNSGK | 11% | 10% | 18 |
| GAPDH | P04406 | G3P | YDNSLK | 13% | 14% | 19 |
| GDA | Q9Y2T3 | GUAD | DHLLGVSDSGK | 17% | 12% | 20 |
| GOT1 | P17174 | AATC | IGADFLAR | 12% | 10% | 21 |
| GSN | P06396 | GELS | AGALNSNDAFVLK | 13% | 11% | 22 |
| KNG1 | P01042 | KNG1 | VQVVAGK | 12% | 12% | 23 |
| L1CAM | P32004 | L1CAM | GQLSFNLR | 14% | 14% | 24 |
| LAMP1 | P11279 | LAMP1 | VWVQAFK | 13% | 13% | 25 |
| LAMP2 | P13473 | LAMP2 | YLDFVFAVK | 19% | 20% | 26 |
| LDHB | P07195 | LDHB | FIIPQIVK | 15% | 14% | 27 |
| MDH1 | P40925 | MDHC | GEFVTTVQQR | 13% | 12% | 28 |
| NCAM1 | P13591 | NCAM1 | GLGEISAASEFK | 12% | 12% | 29 |
| NPTX2 | P47972 | NPTX2 | VAELEDEK | 8% | 10% | 30 |
| NPTXR | O95502 | NPTXR | ELDVLQGR | 8% | 8% | 31 |
| NRXN1 | P58400 | NRX1B | LAIGFSTVQK | 15% | 13% | 32 |
| OGN | P20774 | MIME | LEGNPIVLGK | 11% | 10% | 33 |
| OMG | P23515 | OMGP | LESLPAHLPR | 13% | 17% | 34 |
| PARK7 | Q99497 | PARK7 | ALVILAK | 15% | 15% | 35 |
| PEBP1 | P30086 | PEBP1 | VLTPTQVK | 11% | 12% | 36 |
| PGLYRP2 | Q96PD5 | PGRP2 | TFTLLDPK | 10% | 10% | 37 |
| PKM | P14618 | KPYM | VVEVGSK | 10% | 14% | 38 |
| PKM2 | Q504U3 | | LFEEL VR | 10% | 8% | 39 |
| PON1 | P27169 | PON1 | LLIGTVFHK | 11% | 10% | 40 |
| PPIA | P62937 | PPIA | VSFELFADK | 20% | 15% | 41 |
| PTPRZ1 | P23471 | PTPRZ | AIIDGVESVSR | 11% | 12% | 42 |
| SCG2 | P13521 | SCG2 | IESQTQEEVR | 15% | 14% | 43 |
| SMOC1 | Q9H4F8 | SMOC1 | AQALEQAK | 17% | 13% | 44 |
| SOD1 | P00441 | SODC | HVGDLGNVTADK | 10% | 14% | 45 |
| SPP1 | P10451 | OSTP | QETLPSK | 15% | 10% | 46 |
| TPI1 | P60174 | TPIS | IAVAAQNCYK | 19% | 18% | 47 |
| VGF | O15240 | VGF | EPVAGDAVPGPK | 11% | 12% | 48 |
| VGF | O15240 | VGF | GLQEAAEER | 10% | 12% | 49 |
| VTN | P04004 | VTNC | GQYCYELDEK | 14% | 12% | 50 |
| YWHAB | P31946 | 1433B | NLLSVAYK | 13% | 10% | 51 |
| YWHAB | P31946 | 1433B | VISSIEQK | 12% | 10% | 52 |
| YWHAZ | P63104 | 1433Z | VVSSIEQK | 18% | 12% | 53 |

APOE Allele Specific Peptides

| | | | | | | |
|---|---|---|---|---|---|---|
| APOE2 | | | CLAVYQAGAR | 12% | ND | 54 |
| APOE4 | | | LGADMEDVR | 30% | 18% | 55 |
| APOE | P02649 | APOE | ELQAAQAR | 12% | 10% | 56 |
| APOE2or3 | P02649 | APOE | LGADMEDVCGR | 13% | 13% | 57 |
| APOE3or4 | P02649 | APOE | LAVYQAGAR | 14% | 12% | 58 |

Background Peptides

| | | | | | | |
|---|---|---|---|---|---|---|
| ALB | P02768 | ALBU | LVNEVTEFAK | 11% | 12% | 59 |
| ALB | P02768 | ALBU | LVTDLTK | 11% | 11% | 60 |
| HBA1 | P69905 | HBA | FLASVSTVLTSK | 10% | 11% | 61 |
| HBB | P68871 | HBB | VNVDEVGGEALGR | 13% | 10% | 62 |

TABLE 4

| | | | | |
|---|---|---|---|---|
| | Protein | | | |
| | Accession | | | |
| Protein Gene | Number | Protein Name | Peptide | SEQ ID NO |

Biomarker Peptides of Interest

| | | | | |
|---|---|---|---|---|
| ALDOA | P04075 | ALDOA | AAQEEYVK | 69 |
| NPTXR | O95502 | NPTXR | ADQDTIR | 70 |
| ALDOA | P04075 | ALDOA | DGADFAK | 71 |
| CALM2 | P0DP24 | CALM2 | DGNGYISAAELR | 72 |
| PTPRZ1 | P23471 | PTPRZ | DIEEGAIVNPGR | 73 |
| LDHB | P07195 | LDHB | DYSVTANSK | 74 |
| KNG1 | P01042 | KNG1 | EGDCPVQSGK | 75 |
| AHSG | P02765 | FETUA | EHAVEGDCDFQLLK | 76 |
| ALDOA | P04075 | ALDOA | ELSDIAHR | 77 |
| MDH1 | P40925 | MDHC | ENFSCLTR | 78 |
| COL6A1 | P12109 | CO6A1 | EPCGGLEDAVNEAK | 79 |
| APOC2 | P02655 | APOC2 | ESLSSYWESAK | 80 |
| MFGE8 | Q08431 | MFGM | EVTGIITQGAR | 81 |
| GMFB | P60983 | GMFB | FIVYSYK | 82 |
| MDH1 | P40925 | MDHC | FVEGLPINDFSR | 83 |
| GAPDH | P04406 | G3P | GALQNIIPASTGAAK | 84 |
| PKM | P14618 | KPYM | GDLGIEIPAEK | 85 |
| SPP1 | P10451 | OSTP | GDSVVYGLR | 86 |
| PKM | P14618 | KPYM | GDYPLEAVR | 87 |
| AMBP | P02760 | AMBP | GECVPGEQEPEPILIPR | 88 |
| PEBP1 | P30086 | PEBP1 | GNDISSGTVLSDYVGSGPPK | 89 |
| CHI3L1 | P36222 | CH3L1 | GNQWVGYDDQESVK | 90 |
| AMBP | P02760 | AMBP | GVCEETSGAYEK | 91 |
| PKM | P14618 | KPYM | GVNLPGAAVDLPAVSEK | 92 |
| THY1 | P04216 | THY1 | HVLFGTVGVPEHTYR | 93 |
| PGAM1 | P18669 | PGAM1 | HYGGLTGLNK | 94 |
| CTSB | P07858 | CATB | ICEPGYSPTYK | 95 |
| GDA | Q9Y2T3 | GUAD | IVFLEEASQQEK | 96 |
| RBP4 | P02753 | RET4 | LIVHNGYCDGR | 97 |
| ITGB2 | P05107 | ITB2 | LLVFATDDGFHFAGDGK | 98 |
| PEBP1 | P30086 | PEBP1 | LYEQLSGK | 99 |
| SCG2 | P13521 | SCG2 | SGOLGIQEEDLR | 100 |
| F2 | P00734 | THRB | TATSEYQTFFNPR | 101 |
| CD44 | P16070 | CD44 | TEAADLCK | 102 |
| CHI3L1 | P36222 | CH3L1 | TLLSVGGWNFGSQR | 103 |
| DTD1 | Q8TEA8 | DTD1 | VFEDESGK | 104 |
| GOT1 | P17174 | AATC | VGNLTVVGK | 105 |
| LDHC | P07864 | LDHC | VIGSGCNLDSAR | 106 |
| MDH1 | P40925 | MDHC | VIVVGNPANTNCLTASK | 107 |
| ENO2 | P09104 | ENOG | VNQIGSVTEAIQACK | 108 |
| SPON1 | Q9HCB6 | SPON1 | VTLSAAPPSYFR | 109 |
| GDI1 | P31150 | GDIA | VVEGSFVYK | 110 |
| TXNRD2 | Q9NNW7 | TRXR2 | WGLGGTCVNVGCIPK | 111 |
| CD44 | P16070 | CD44 | YGFIEGHVVIPR | 112 |
| ENO1 | P06733 | ENOA | YISPDQLADLYK | 113 |
| YWHAG | P61981 | 1433G | YLAEVATGEK | 114 |
| YWHAB | P31946 | 1433B | YLIPNATQPESK | 115 |
| PEBP1 | P30086 | PEBP1 | YVWLVYEQDRPLK | 116 |

### Monitoring LC-MS/MS Instrument Performance

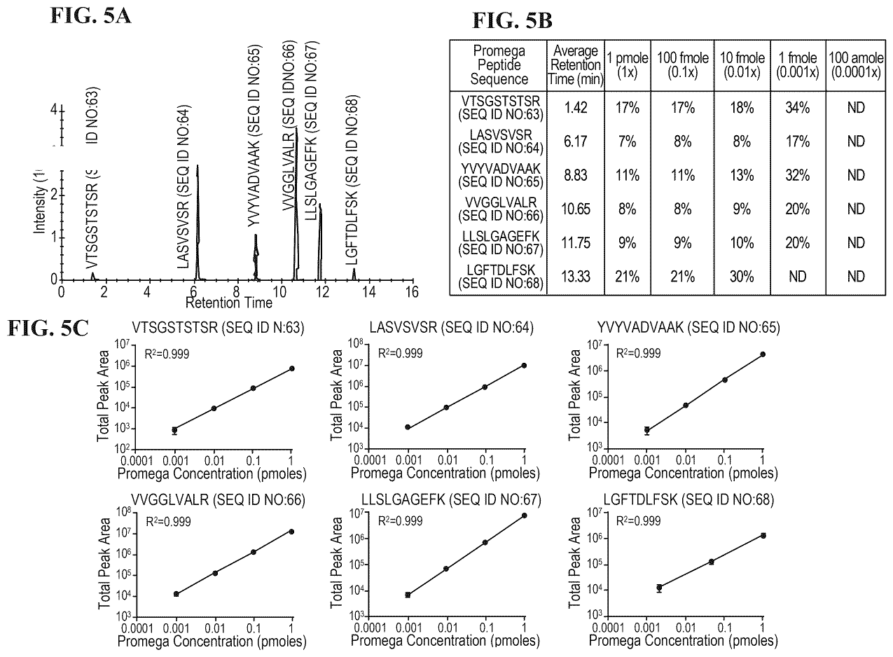

The sample reconstitution solution contained Promega 6×5 LC-MS/MS Peptide Reference Mix (50 fmole/μL). The Promega Peptide Reference Mix20 provides a convenient way to assess LC column performance and MS instrument parameters, including sensitivity and dynamic range. The mix consists of 30 peptides; 6 sets of 5 isotopologues of the same peptide sequence, differing only in the number of stable, heavy-labeled amino acids incorporated into the sequence using uniform ¹³C and ¹⁵N atoms making them chromatographically indistinguishable. The isotopologues were specifically synthesized to cover a wide range of hydrophobicities so that dynamic range could be assessed across the gradient profile (FIG. 5A). Each isotopologue represents a series of tenfold dilutions, estimated to be 1 pmole, 100 fmole, 10 fmole, 1 fmole, and 100 amole for each peptide sequence in a 20 μL injection, a range that would challenge the lowest limits of detection of the method (FIG. 5B). We assessed the raw peak areas in 423 injections over 5 days to determine the label-free CV for each peptide isotopologue (FIG. 5B). The 100 amole level (0.0001×) was not detected (ND) for any of the peptide sequences. Based on the label-free CV, the lowest limit of detection was determined for each peptide to be between 1-10 fmole across the gradient profile with a dynamic range spanning 4 orders of magnitude for all peptides except the latest eluting peptide at 13.3 minutes (FIG. 5C).

### Technical Replicate Variance

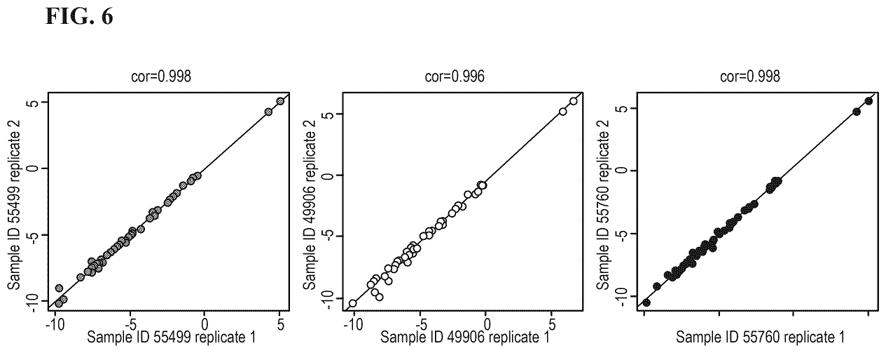

Three individual samples were analyzed in duplicates scattered throughout the sample run sequence to assess technical replicate variance. The log 2 (ratio) was graphed for each of 58 biomarker peptides in replicate 1 (x-axis) versus replicate 2 (y-axis) and the Pearson correlation coefficient was determined (FIG. 6). The analysis showed a near-identical correlation (ρ=0.996-0.998) between each of the technical replicate pairs for the three individual CSF samples, supporting the same high level of method reproducibility we found using the QC pools. In contrast, the mean correlation of the same 58 log 2 (ratios) for all 390 non-replicate samples to those of each of the other 389 non-replicate samples' log 2 (ratios) averaged p=0.96 for 151,710 correlations, which was significantly lower.

### Example 2

Concordance Between a Discovery (ssTMT) and Replication (SRM) Datasets

Given that the peptide targets were largely based on multiple single-shot tandem mass tag (ssTMT) dataset, a comparison between the ssTMT identified peptides and the SRM identified peptides was performed using one of the ssTMT datasets comprising of 297 individuals (147 control and 150 AD). Fourth-four (44) of 62 SRM peptides overlapped with this ssTMT dataset. In addition, for 40 of the overlapping peptides, significant correlation (cor=0.91; p=2.8⁻¹⁵) between SRM and ssTMT peptides was observed, highlighting the accuracy and concordance of measurements across both MS assays. Thus, despite substantial differences in chromatography (nanoflow versus standard flow), MS instrumentation (Orbitrap versus triple quadrupole), and protein quantitation approaches (ssTMT versus SRM), the selected peptides in this assay were highly reproducible and robust in their direction of change in AD CSF. Furthermore, the enhanced throughput of the SRM protocol (96 samples per day) allowed for the examination of large cohorts relatively quickly as compared to previously published unbiased discovery proteomics and parallel reaction monitoring experiments.

### Example 3

### Stage-Specific Differences in Peptide and Protein Levels

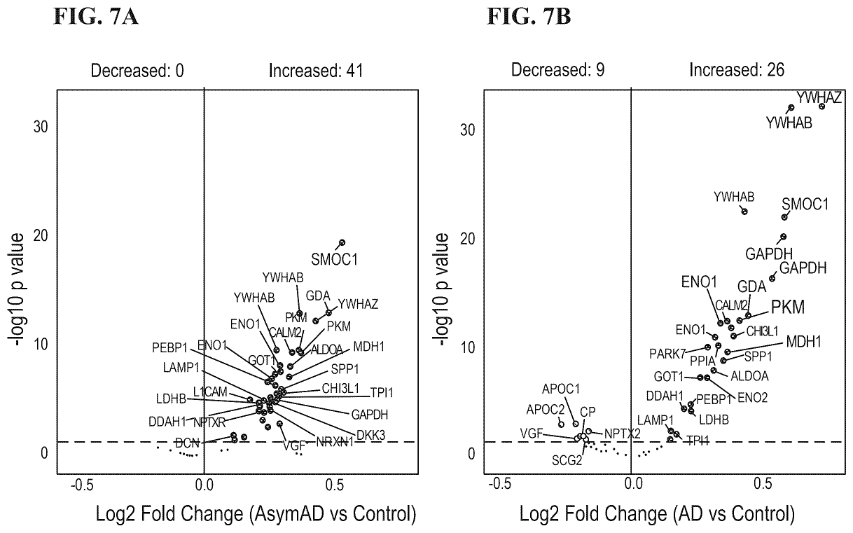

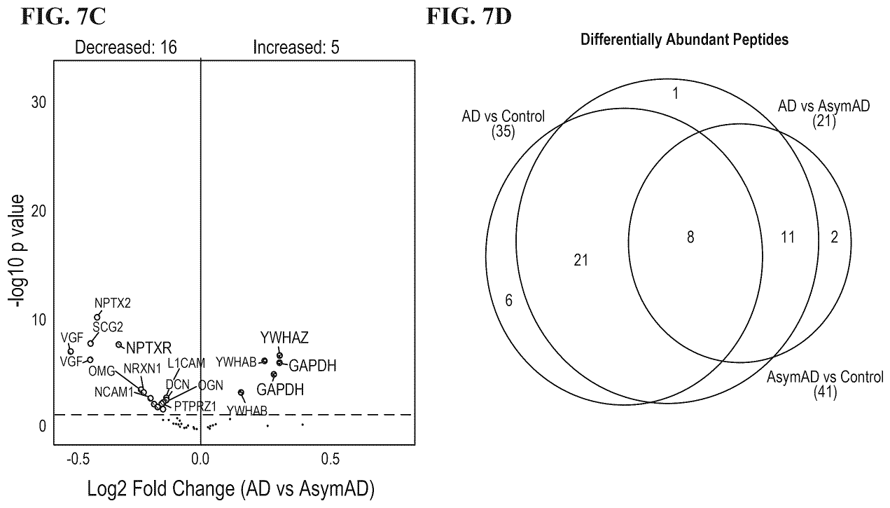

The described cohort included control, AD, and AsymAD groups across the Amyloid/Tau/Neurodegeneration (AT/N) framework, which allows for the comparison of peptide and protein differential abundance across stages of disease. Comparisons that were specific to symptomatic AD or those with potential for staging AD by using the preclinical, AsymAD, group compared to the control group was performed. By comparing candidate biomarkers using ANOVA (excluding APOE allele-specific peptides), 41 differentially expressed peptides (36 proteins) in AsymAD vs controls (FIG. 7A), 35 differentially expressed peptides (30 proteins) in AD versus controls (FIG. 7B), and 21 differentially expressed peptides (18 proteins) in AD vs AsymAD (FIG. 7C). The Venn diagram summarizes the differentially expressed peptides across groups in FIG. 7D.

### Example 4

Stratifying Early from Progressive Biomarkers of AD

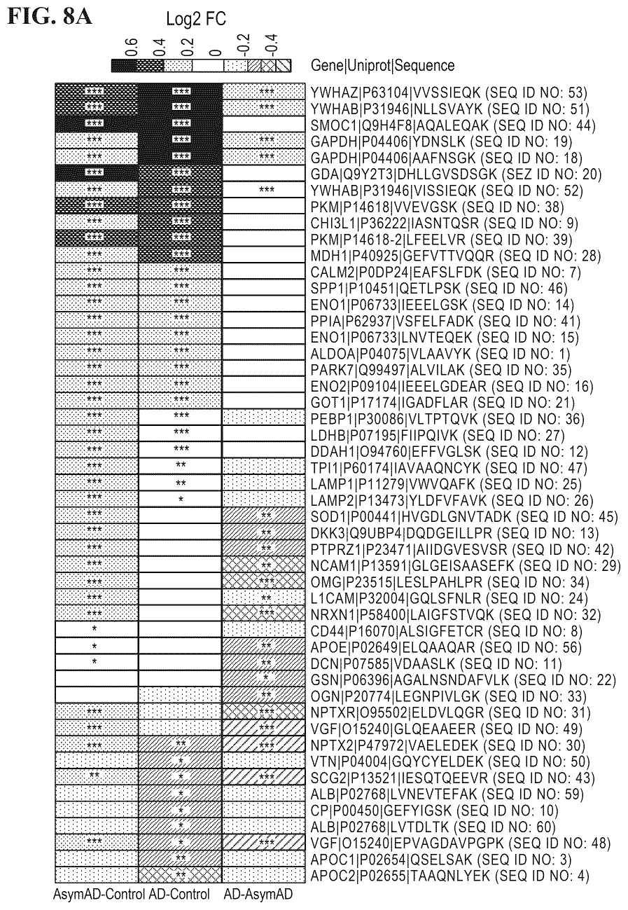

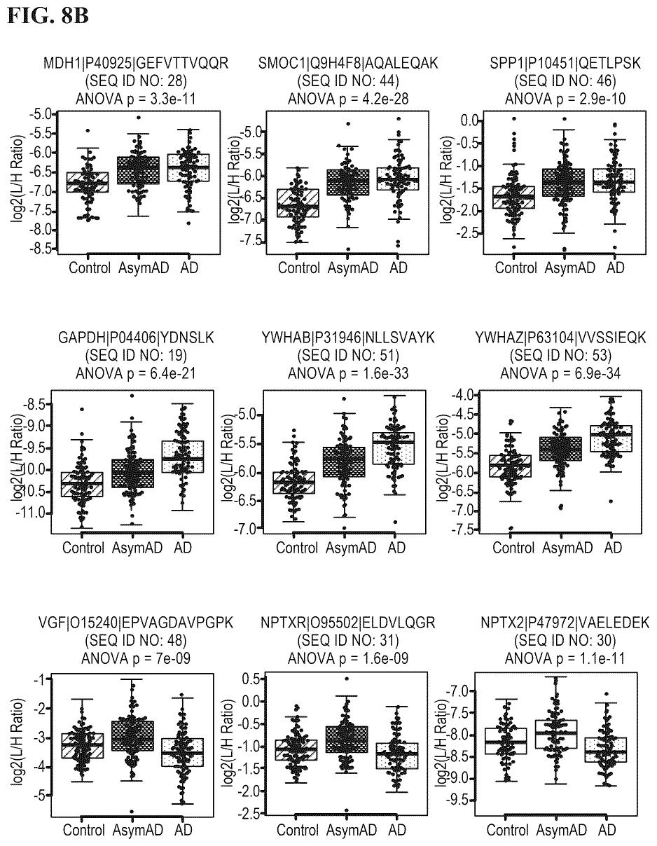

Using a differential abundance analysis, the changing proteins were stratified as early or progressive biomarkers of AD (FIGS. 7 and 8). The log 2-fold change (Log 2 FC) from the volcano plots in FIGS. 7A-7D are represented as a heatmap in FIG. 8A to illustrate how each peptide is changing across each group comparison. Twenty-two peptides (21 proteins) were early biomarkers of AD because they were significantly different in AsymAD versus controls but not significantly different in AD versus AsymAD (FIG. 8A). A plurality of these proteins mapped to metabolic enzymes linked to glucose metabolism (PKM, MDH1, ENO1, ALDOA, ENO2, LDHB, and TPI1). SMOC1 and SPP1, markers linked to glial biology and inflammation were also increased in AsymAD samples compared to controls (FIG. 8B, top row). GAPDH, YWHAB and YWHAZ proteins were found to be progressive biomarkers of AD because the proteins were differentially expressed from Control to AsymAD and from AsymAD to AD with a consistent trend in direction of change (FIG. 8B, middle row). Proteins associated with neuronal/synaptic markers including VGF, NPTX2, NPTXR, and L1CAM were increased in AsymAD compared to controls but decreased in AD vs controls (FIG. 8B, lower row). As noted above, these proteins could play a role in cognitive resilience, as these are some of the most strongly correlated to slope of cognitive decline in human brain proteome studies. Interestingly, 14 peptides (13 proteins) that were up in AsymAD as compared to Control but down in AD when compared to AsymAD were identified. A majority of these proteins map to neuronal/synaptic markers including VGF, NPTX2, NPTXR, and L1CAM among others, suggesting that these proteins could play a role in cognitive resilience as these are some of the most strongly correlated to slope of cognitive decline in human brain proteome studies.

### Example 5

### Receiver-Operating Characteristic (ROC) Analysis for Evaluating Biomarker Diagnostic Capability

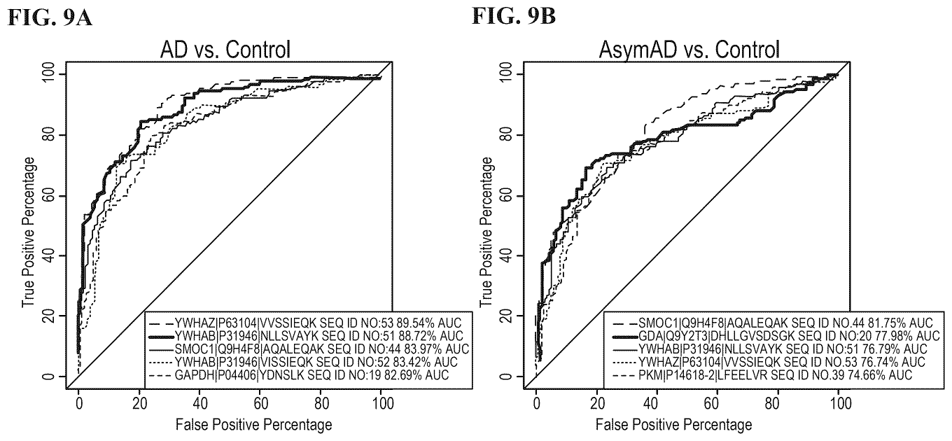

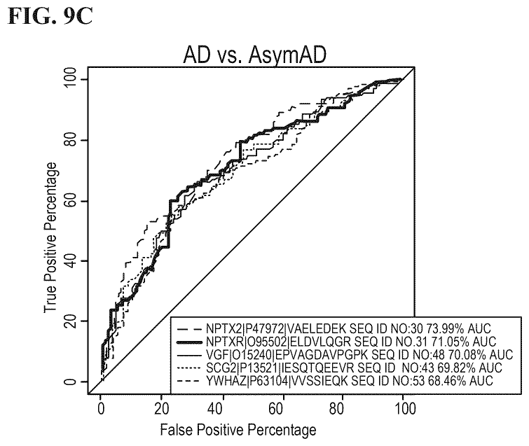

The capacity for peptide measurements to serve as diagnostic biomarkers distinguishing individuals with AD and even asymptomatic disease from individuals not on a trajectory to develop AD is well-established, with secreted amyloid and tau peptide measurements in CSF being the current gold standard for interrogation of patients' AD stage from their CSF where CSF amyloid beta peptide concentration inversely correlates to plaque deposition in the living brain. The measurements of additional peptides collected here are appropriate for comparison to the ELISA measurements of CSF amyloid and Tau biomarker positivity, or a dichotomized cognition rating, or other ancillary traits such as diagnosis for the 390 individuals can be performed. To demonstrate this utility, ROC curve analysis was performed and the area under the curve (AUC) was calculated for all 62 precision peptide measures as fitting a logistic regression to 3 subsets of case samples divided to represent known pairs of diagnoses, namely AD versus control, AsymAD versus control, and AD vs AsymAD (FIGS. 9A-9C). The top performing peptide for the YWHAZ gene product 14-3-3 ζ (protein demonstrated an AUC of 89.5% discrimination of AD from control cases. SMOC1 AUC of 81.8% was the best performing peptide for discrimination of AsymAD from control case samples, and NPTX2 had an AUC of 74.0% in the AD versus AsymAD in contrast. FIGS. 9A-9C show the top five peptides by AUC for each of the three comparisons, highlighting the potential of this data set to aid in the design or validation of diagnostic biomarkers. Additional analysis for combinatorial, multi-peptide biomarkers using these data to diagnose, subclassify, predict disease onset, and gauge treatment efficacy are called for in future studies.

### Example 6

### CSF and Brain Proteomics of Diverse Cohorts

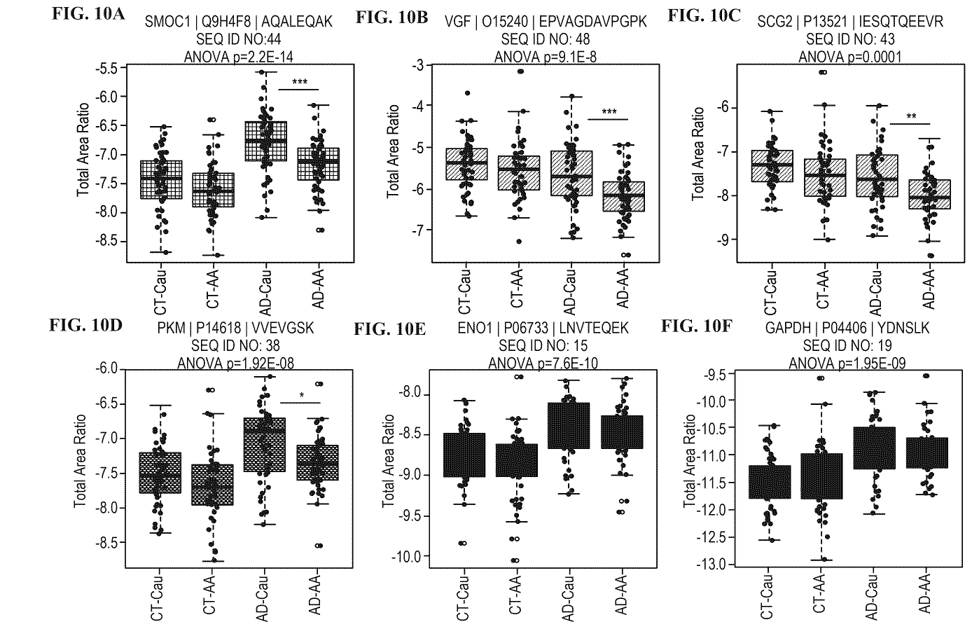

Chronic health conditions including cardiovascular disease and diabetes, lower quality and level of education, higher rates of poverty, and greater exposure to discrimination disproportionally affect African Americans, putting this population at a heightened risk for Alzheimer's disease (AD) and related dementias. Thus, a SRM-MS targeted proteomic study of CSF was performed to define proteins that are similar or divergent in African Americans and Caucasians with AD. To this end, the panel of selected peptides identified in Example 1 were measured as described above in a balanced cohort of African American and Caucasian CSF samples, matched for age, sex, and diagnosis from the Emory ADRC. This included 53 Caucasian Controls, 52 African American Controls, 48 AD Caucasians, and 51 AD African Americans. Results are shown in FIGS. 10A-10F.

Peptides measuring SMOC1 and PKM are increased in AD in both African Americans and Caucasians. However, SMOC1 and PKM levels are significantly lower in African Americans with AD compared to Caucasian with AD. In contrast, peptides quantifying neuronal markers VGF and SCG1 are decreased in AD in both races. Levels of VGF and SCG2, however, are significantly lower in African Americans with AD compared to Caucasians. ENO1 and GAPDH are increased proportionally in both African and Caucasian populations and do not diverge by race.

### Example 7

### CSF Peptide Biomarkers of Treatment Response and Target Engagement

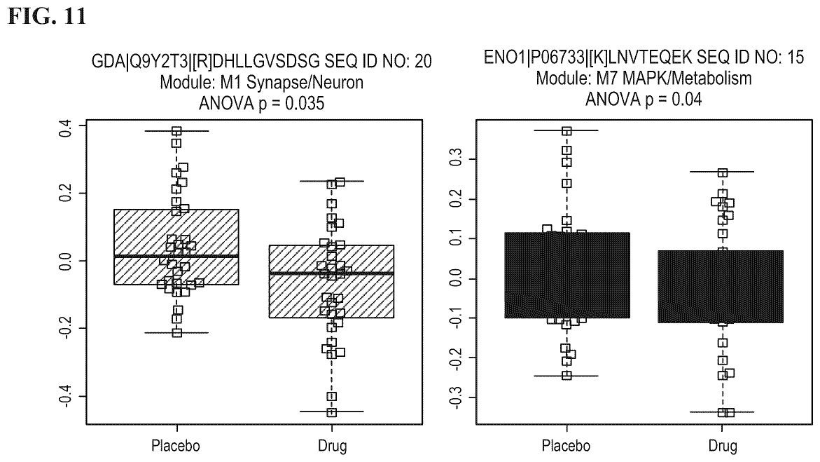

An important unmet goal in the field is the ability to predict treatment response and target engagement. The ability of the identified CSF peptides to serve these purposes using CSF samples obtained in a clinical trial of atomoxetine (ATX) in subjects with mild cognitive impairment (MCI) was tested. ATX is an FDA-approved norepinephrine (NE) transporter inhibitor used clinically for attention disorders. The trial was performed at the Goizueta Alzheimer's Disease Research Center (ADRC) to test the therapeutic hypothesis that ATX is safe and well tolerated, achieves target engagement, and reduces CNS inflammation. The study was designed as a single-center double-blind crossover trial, in which MCI patients with prodromal AD (confirmed by CSF AD biomarkers Aβ42, Tau, and P-Tau181) were randomized to ATX/placebo and placebo/ATX treatment arms. To establish proof of concept of our novel CSF peptide biomarkers for assessing drug engagement and treatment response, the CSF peptide biomarkers were analyzed in samples at baseline and after 6 months of either placebo (n=31) or ATX (n=31). Notably, a biomarker response was observed for individual protein biomarkers (FIG. 11). The peptides can also be grouped by their brain co-expression patterns that reflect synaptic, myelination, glial immunity, vascular, and metabolic panels. While little differences were observed in the vascular panel with ATX treatment, participants with prodromal AD that received the ATX treatment showed an increase in the myelination and glial immunity panels compared to placebo and non-treated AD patients and decreases in the abundance of the metabolic and synaptic panels. These data highlight the utility of these CSF proteins individually or as groups as biomarker panels for establishing a treatment response, and for identifying the types of responses for a given drug and individual.

### Example 8

Machine Learning to Identify CSF Peptides that Individually and Collectively Best Inform Various Traits and Endophenotypes (e.g., Diagnosis, Preclinical AD Status, Disease Staging and Progression, Cognitive Decline, Brain Atrophy)

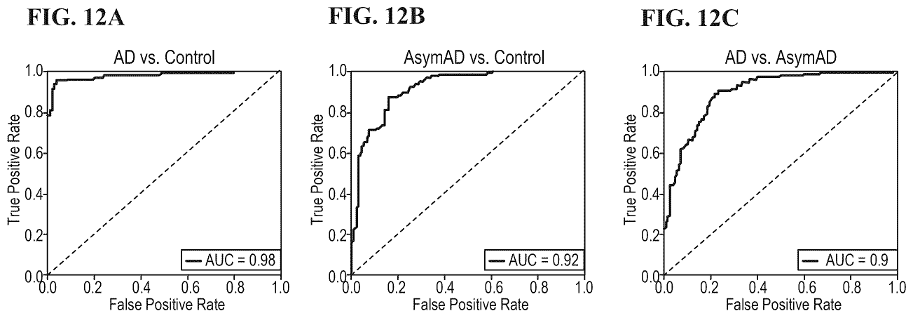

An important unmet goal in the field is the ability of CSF measures to accurately serve as biomarkers for a range of clinical and research needs. Using machine learning and explainable AI, examples demonstrating performance of a panel of peptide biomarkers in classifying AD cases from controls, Asymptomatic AD cases from controls (i.e., identifying AD pathology in cognitively intact controls), and AD cases progressing from Asymptomatic AD) were run. As seen in FIGS. 12A-12C, an optimal combination of the panel of peptides determined by machine learning were more accurate than the 5 highest performing individual peptides shown FIGS. 9A-9C. For example, the top performing peptide for discriminating AD from the control case, the YWHAZ gene product 14-3-3 (protein, displayed an AUC of 90%, while the AUC of the protein panel is 98%. Similarly, SMOC1, the best performing peptide for discrimination of AsymAD from control case samples, had an AUC of 81%, while the panel of peptides achieved AUCs of 92%. This trend continued for the top performing peptide discriminating between AD versus AsymAD, NPTX2, which displayed an AUC of 74.0%, while the panel of peptides achieved an AUC of 90%.

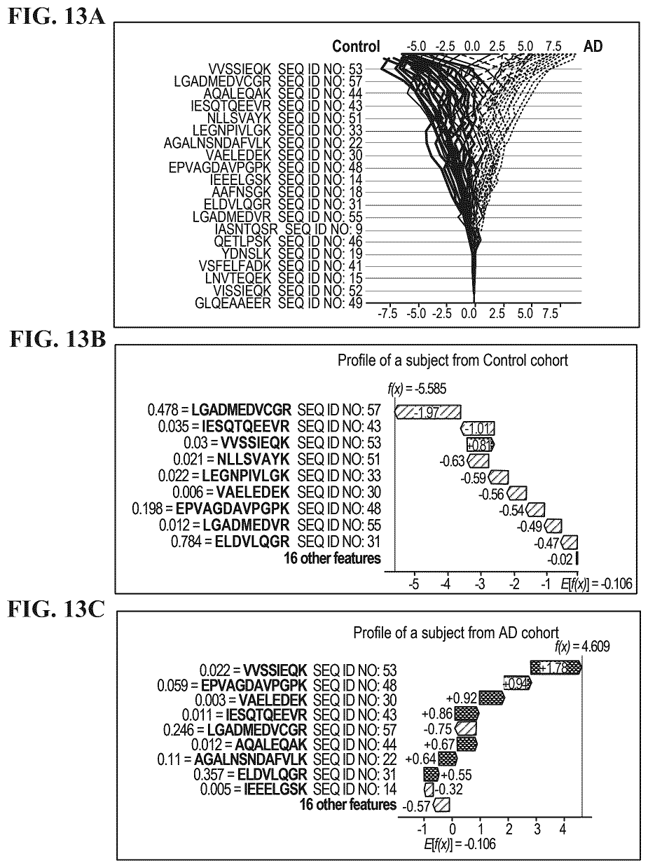

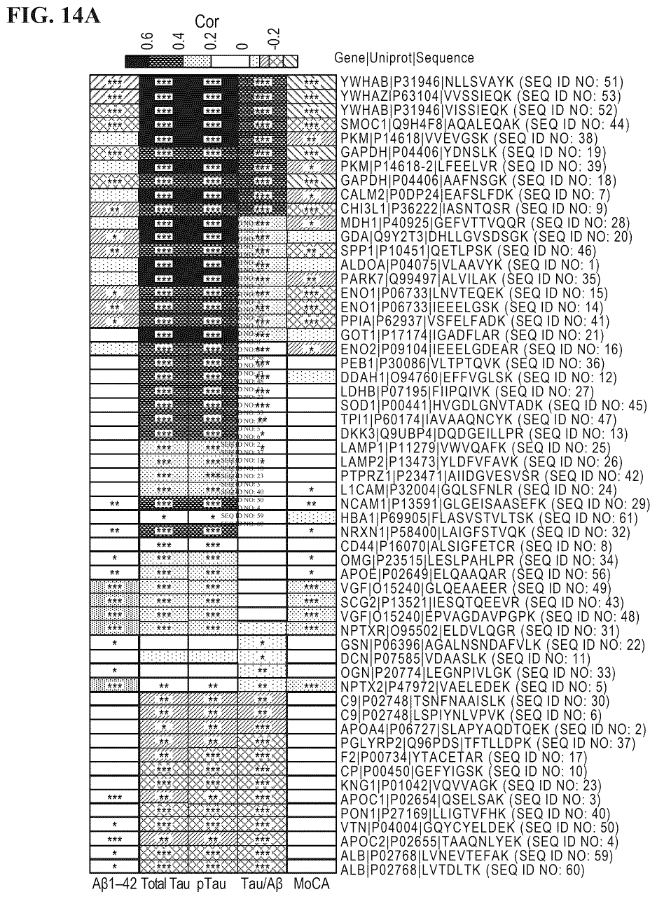

Using SHAP47 analysis (see Lundberg et al. From local explanations to global understanding with explainable AI for trees. Nature Machine Intelligence 2020, 2:56-67) as shown in FIG. 13A, the relative contribution of individual peptides in a panel was evaluated toward the final decision of assigning a diagnosis into one of the three categories—Control, AD, AsymAD. Further, as the contribution of the same peptide varied across individuals in the same cohort, the SHAP values of each peptide was used for each individual to create a personalized profile indictive of the person's endophenotype. See FIG. 13B for an exemplary profile from a control subject and FIG. 13C for an exemplary profile from an AD subject.

### Example 9

Correlation of Peptide Biomarker Abundance to Amyloid, Tau, pTau and Cognitive Measurements

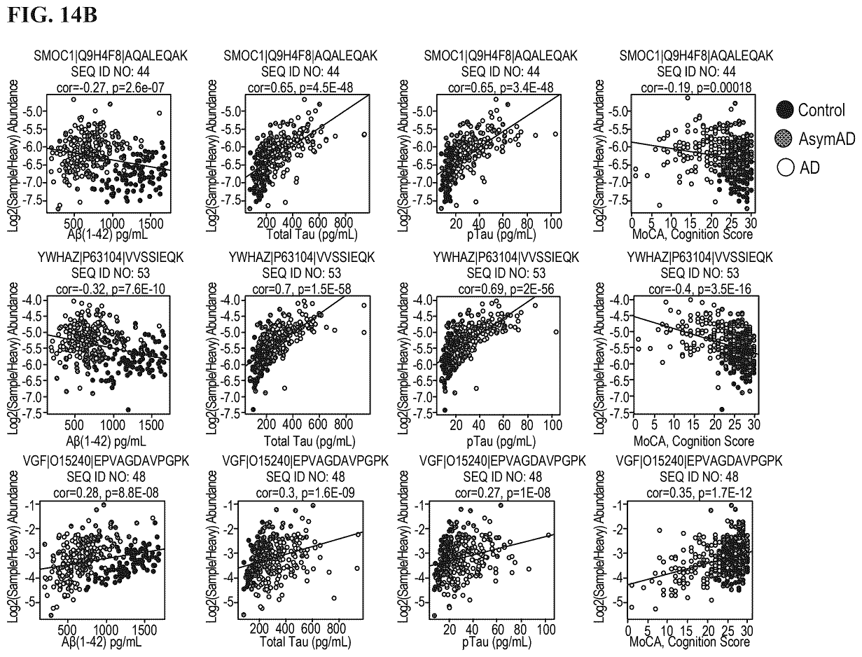

The comparison of existing biomarkers to the SRM peptide measurements can be accomplished by correlation, wherein the degree of correlation indicates how similar a peptide measurement is to the established immunoassay measures of Aβ(1-42), total Tau, and phospho-T181 Tau as well as cognition (MoCA cognition test). In FIG. 14A, 57 of the 58 biomarker peptides have significant absolute correlation to at least one of the above biomarkers or to the ratio of total Tau/amyloid. Correlation to cognition measured by MoCA was also shown. Individual correlation scatterplots and linear fit lines for three of the peptides (SMOC1 AQALEQAK (SEQ ID NO:44), YWHAZ VVSSIEQK (SEQ ID NO:53), and VGF EPVAGDAVPGPK (SEQ ID NO:48) are provided in FIG. 14B. Significant correlations of these peptides to the established biomarker and cognitive measures show that these measurements can be used to explain variance between specific biomarkers distinct from amyloid and Tau measurements and to classify or stage disease progression.

The products and methods of the appended claims are not limited in scope by the specific products and methods described herein, which are intended as illustrations of a few aspects of the claims and any dispersions, products, and methods that are functionally equivalent are intended to fall within the scope of the claims. Various modifications of the products and methods in addition to those shown and described herein are intended to fall within the scope of the appended claims. Further, while only certain representative materials and method steps disclosed herein are specifically described, other combinations of the materials and method steps also are intended to fall within the scope of the appended claims, even if not specifically recited. Thus, a combination of steps, elements, components, or constituents may be explicitly mentioned herein: however, other combinations of steps, elements, components, and constituents are included, even though not explicitly stated.

1. A method for measuring multiple peptides indicative of cognitive function in a cerebrospinal fluid or plasma sample from a subject comprising
(a) treating the cerebrospinal fluid or plasma sample from the subject with trypsin to produce a peptide solution comprising multiple peptides indicative of cognitive function, wherein the multiple peptides indicative of cognitive function comprise two or more of the peptides having the amino acid sequence of SEQ ID NO:1-53 and SEQ ID NO:69-116;
(b) adding to the peptide solution a reference standard comprising isotopically labeled peptides to produce a test solution;
(c) detecting the multiple peptides indicative of cognitive function and the isotopically labeled peptides in the test solution using selective reaction monitoring-based mass spectrometry; and
(d) determining an amount of the multiple peptides indicative of cognitive function.

2. The method of claim 1, wherein the multiple peptides indicative of cognitive function comprise two or more peptides selected from the group consisting of
(SEQ ID NO: 18)
AAFNSGK,
(SEQ ID NO: 22)
AGALNSNDAFVLK:,
(SEQ ID NO: 35)
ALVILAK,
(SEQ ID NO: 44)
AQALEQAK;
(SEQ ID NO: 20)
DHLLGVSDSGK,
(SEQ ID NO: 7)
EAFSLFDK,
(SEQ ID NO: 31)
ELDVLQGR,
(SEQ ID NO: 48)
EPVAGDAVPGPK,
(SEQ ID NO: 49)
GLQEAAEER,
(SEQ ID NO: 24)
GQLSFNLR,
(SEQ ID NO: 9)
IASNTQSR,
(SEQ ID NO: 18)
IEEELGSK,
(SEQ ID NO: 43)
IESQTQEEVR,
(SEQ ID NO: 32)
LAIGFSTVQK,
(SEQ ID NO: 33)
LEGNPIVLGK,
(SEQ ID NO: 39)
LFEELVR,
(SEQ ID NO: 15)
LNVTEQEK,
(SEQ ID NO: 51)
NLLSVAYK,
(SEQ ID NO: 46)
QETLPSK,
(SEQ ID NO: 3)
QSELSAK,
(SEQ ID NO: 30)
VAELEDEK,
(SEQ ID NO: 52)
VISSIEQK,
(SEQ ID NO: 19)
YDNSLK,
and
(SEQ ID NO: 53)
VVSSIEQK.

3. The method of claim 2, wherein the multiple peptides indicative of cognitive function comprise VISSIEQK (SEQ ID NO:52), VVSSIEQK (SEQ ID NO:53), and NLLSVAYK (SEQ ID NO:51).

4. The method of claim 1, wherein the multiple peptides indicative of cognitive function comprise two or more peptides selected from the group consisting of
(SEQ ID NO: 69)
AAQEEYVK,
(SEQ ID NO: 70)
ADQDTIR,
(SEQ ID NO: 71)
DGADFAK,
(SEQ ID NO: 72)
DGNGYISAAELR,
(SEQ ID NO: 73)
DIEEGAIVNPGR,
(SEQ ID NO: 74)
DYSVTANSK,
(SEQ ID NO: 75)
EGDCPVQSGK,
(SEQ ID NO: 76)
EHAVEGDCDFQLLK,
(SEQ ID NO: 77)
ELSDIAHR,
(SEQ ID NO: 78)
ENFSCLTR,
(SEQ ID NO: 79)
EPCGGLEDAVNEAK,
(SEQ ID NO: 80)
ESLSSYWESAK,
(SEQ ID NO: 81)
EVTGIITQGAR,
(SEQ ID NO: 82)
FIVYSYK,
(SEQ ID NO: 83)
FVEGLPINDFSR,
(SEQ ID NO: 84)
GALQNIIPASTGAAK,
(SEQ ID NO: 85)
GDLGIEIPAEK,
(SEQ ID NO: 86)
GDSVVYGLR,
(SEQ ID NO: 87)
GDYPLEAVR,
(SEQ ID NO: 88)
GECVPGEQEPEPILIPR,
(SEQ ID NO: 89)
GNDISSGTVLSDYVGSGPPK,
(SEQ ID NO: 90)
GNQWVGYDDQESVK,
(SEQ ID NO: 91)
GVCEETSGAYEK,
(SEQ ID NO: 92)
GVNLPGAAVDLPAVSEK,
(SEQ ID NO: 93)
HVLFGTVGVPEHTYR,
(SEQ ID NO: 94)
HYGGLTGLNK,
(SEQ ID NO: 95)
ICEPGYSPTYK,
(SEQ ID NO: 96)
IVFLEEASQQEK,
(SEQ ID NO: 97)
LIVHNGYCDGR,
(SEQ ID NO: 98)
LLVFATDDGFHFAGDGK,
(SEQ ID NO: 99)
LYEQLSGK,
(SEQ ID NO: 100)
SGQLGIQEEDLR,
(SEQ ID NO: 101)
TATSEYQTFFNPR,
(SEQ ID NO: 102)
TEAADLCK,
(SEQ ID NO: 103)
TLLSVGGWNFGSQR,
(SEQ ID NO: 104)
VFEDESGK,
(SEQ ID NO: 105)
VGNLTVVGK,
(SEQ ID NO: 106)
VIGSGCNLDSAR,
(SEQ ID NO: 107)
VIVVGNPANTNCLTASK,
(SEQ ID NO: 108)
VNQIGSVTEAIQACK,
(SEQ ID NO: 109)
VTLSAAPPSYFR,
(SEQ ID NO: 110)
VVEGSFVYK,
(SEQ ID NO: 111)
WGLGGTCVNVGCIPK,
(SEQ ID NO: 112)
YGFIEGHVVIPR,
(SEQ ID NO: 113)
YISPDQLADLYK,
(SEQ ID NO: 114)
YLAEVATGEK,
(SEQ ID NO: 115)
YLIPNATQPESK,
and
(SEQ ID NO: 116)
YVWLVYEQDRPLK.

5. The method of claim 1, wherein the isotopically labeled peptides comprise peptides having the amino acid sequences of SEQ ID NO:63-68 with labeled C-terminal lysine or arginine residues.

6. The method of claim 1, further comprising identifying an Alzheimer's Disease state in the subject.

7. The method of claim 6, wherein the Alzheimer's disease state of the subject is Alzheimer's Disease positive or Alzheimer's Disease negative.

8. The method of claim 7, wherein the detected peptides indicative of Alzheimer's Disease positive state comprise peptide fragments of glucose metabolism enzymes.

9. The method of claim 6, wherein the glucose metabolism enzymes comprise PKM, MDH1, ENO1, ALDOA, ENO2, LDHB, and TPI1.

10. The method of claim 7, wherein the detected peptides indicating the subject is Alzheimer's Disease positive comprise;
a) two or more of the peptides having the amino acid sequence of SEQ ID NO:44, SEQ ID NO:20, SEQ ID NO:39, SEQ ID NO:57, SEQ ID NO:55, SEQ ID NO:51, SEQ ID NO:53 SEQ ID NO:52, or SEQ ID NO:19;
b) SEQ ID NO:53, SEQ ID NO: 51, SEQ ID NO:44, SEQ ID NO:52, and SEQ ID NO:19; or
c) SEQ ID NO: 96 and SEQ ID NO: 115.

11-12. (canceled)

13. The method of claim 6, wherein the Alzheimer's Disease state of the subject is asymptomatic Alzheimer's Disease.

14. The method of claim 13, wherein the detected peptides indicative of asymptomatic Alzheimer's Disease state comprise:
a) two or more peptides having the amino acid sequence of SEQ ID NO:44, SEQ ID NO:55, SEQ ID NO:20, SEQ ID NO:51, or SEQ ID NO:53;
b) peptides having the amino acid sequence of SEQ ID NO:44, SEQ ID NO:55, SEQ ID NO:20, SEQ ID NO:51, or SEQ ID NO:53; or
c) SEQ ID NO: 96 and SEQ ID NO: 115.

15-16. (canceled)

17. The method of claim 6, wherein the Alzheimer's Disease state of the subject is symptomatic Alzheimer's Disease.

18. The method of claim 17, wherein the detected peptides indicative of symptomatic Alzheimer's Disease state comprise:
a) two or more peptides having the amino acid sequence of SEQ ID NO:30, SEQ ID NO:31, SEQ ID NO:48, SEQ ID NO:43, or SEQ ID NO:53;
b) peptides having the amino acid sequence of SEQ ID NO:30, SEQ ID NO:31, SEQ ID NO:48, SEQ ID NO:43, or SEQ ID NO:53; or
c) SEQ ID NO: 70 and SEQ ID NO: 100.

19-20. (canceled)

21. The method of claim 6, wherein the Alzheimer's Disease state is further characterized as AT+ or AT−.

22. The method of claim 6, further comprising genotyping the subject positive for Alzheimer's Disease by detecting one or more peptides indicative of apolipoprotein E (APOE), albumin (ALB), hemoglobin subunit A (HBA), or hemoglobin subunit B (HBB) expression in the test solution, wherein detecting the one or more peptides indicative of APOE, ALB, HBA, or HBB expression is performed concurrently by selective reaction monitoring-based mass spectrometry.

23. The method of claim 22, wherein the method further comprises detecting one or more peptides indicative of APOE expression and wherein the APOE expression comprises expression of one or more of APOE2, APOE3, or APOE4.

24. The method of claim 23, wherein the one or more peptides indicative of APOE expression comprise one or more peptides having the amino acid sequence of SEQ ID NO:54-58.

25. The method of claim 1, wherein the multiple peptides indicative of cognitive function are selected using Shapley Additive explanations (SHAP) from the peptides having the amino acid sequence of SEQ ID NO:1-53 and SEQ ID NO:69-116 in the biological sample of the subject.

26. The method of claim 6, wherein the amount of the multiple peptides indicative of cognitive function is corrected for racial differences in expression of selected peptides.

27. The method of claim 26, wherein the selected peptides comprise one or more of peptide fragments of SMOC1, PKM, VGF, SCG1, or SCG2.

28. A method of treating a subject with Alzheimer's Disease comprising
(a) performing the method of claim 6 to select a subject positive for Alzheimer's disease and
(b) administering a therapeutic agent to the subject positive for Alzheimer's Disease.

29. The method of claim 28, further comprising repeating the method of claim 6 following the step of administering the therapeutic agent to determine the efficacy of the therapeutic agent.

30. The method of claim 28, wherein the method comprises detecting one or more of peptide fragments of SMOC1, PKM, VGF, SCG1, or SCG2.

31. A kit comprising peptides having the amino acid sequences of SEQ ID NO:63-68 with isotopically labeled C-terminal lysine or arginine residues.
